(19) United States
(12) Patent Application Publication
(10) Pub. No.: US 2025/0290915 A1
(43) Pub. Date: Sep. 18, 2025

(54) METHOD FOR PREDICTING RESPONSIVENESS TO THERAPY

(71) Applicant: CANCERTAIN LIMITED, Manchester (GB)

(72) Inventors: Himanshu KATARIA, Manchester (GB); Meghan HARRISON, Manchester (GB)

(21) Appl. No.: 18/861,159

(22) PCT Filed: Apr. 27, 2023
(86) PCT No.: PCT/GB2023/051122

§ 371 Date: Oct. 28, 2024

(30) Foreign Application Priority Data
Apr. 7, 2022 (GB) GB2206124.6

(51) Int. Cl. G01N 33/50

(52) U.S. Cl. CPC G01N 33/5011

(57) ABSTRACT

The present invention relates to methods of predicting responsiveness of a subject to a therapeutic agent. In particular, the present invention relates to methods of predicting responsiveness of a subject having cancer to one or more immunotherapeutic agent and one or more chemotherapeutic agent, as well as kits for carrying out such methods. Methods of selecting a subject with cancer for treatment, methods of selecting an immunotherapeutic agent and/or chemotherapeutic agent for use in treatment, and methods of treatment are also provided.

### FIELD OF THE INVENTION

The present invention relates to methods of predicting responsiveness of a subject to a therapeutic agent. In particular, the present invention relates to methods of predicting responsiveness of a subject having cancer to one or more immunotherapeutic agent and one or more chemotherapeutic agent, as well as kits for carrying out such methods. Methods of selecting a subject with cancer for treatment, methods of selecting an immunotherapeutic agent and/or chemotherapeutic agent for use in treatment, and methods of treatment are also provided.

### BACKGROUND OF THE INVENTION

Cancer is one of the leading causes of death in the developed world, with approximately 1.8 million people diagnosed with cancer and over 600,000 deaths per year in the United States alone. Overall it is estimated that more than 1 in 2 people will develop some form of cancer during their lifetime, according to forecast data from Cancer Research UK. In addition to the often devastating impact a cancer diagnosis can have on an individual and their family, cancer has a significant societal impact, particularly in terms of financial costs. Estimated national expenditures for cancer care in the United States in 2018 were $150.8 billion, and costs are likely to increase with an aging population and increasing incidence of cancer. In addition, costs are also likely to increase as new, and often more expensive, treatments are adopted as standards of care.

To maximise the probability of a successful treatment outcome and to minimising costs to the health care system, it is important to ensure that patients are given the correct treatment for their particular cancer. Personalised medicine, based on a patient's specific disease and genes allow for cancers to be targeted more effectively, and with potentially fewer side effects. However, identifying which course of treatment will be most effective for any given patient is not always straightforward. There are more than 200 different types of cancer, four of which-breast, lung, bowel, and prostate, which account for more than half of all new cases in the United Kingdom. Furthermore, even one cancer type, such as breast cancer, is actually a collection of multiple different diseases, with different cell types involved, and different gene mutations. These complexities mean that at present, there is no toolbox or set of diagnostic/screening techniques or assays that allow medical practitioners to readily identify personalised tumour treatments for cancer patients, particularly not at the scale required to make this viable in a clinical setting. Existing tests are costly and time-consuming. Furthermore, using conventional screening methods a significant proportion of cancer patients are prescribed the wrong, or a sub-optional, treatment. Depending on what treatment a patient receives, these incorrect treatments may be ineffective, or even harmful to the patient.

The present invention overcomes one or more of the above-mentioned problems. In particular, it is an object of the present invention to provide a screening method that enables diagnostic matching across all cancer therapies, enabling personalisation of treatment with high diagnostic accuracy. It is also an object of the invention to reduce costs and the time taken to identify the optimal treatment for any given patient. Increasing accuracy and decreasing time for screening can improve outcomes for patients, and together with reduced costs offer benefits to the health care system.

### SUMMARY OF THE INVENTION

The present inventors are the first to develop an assay which allows for different immunotherapeutic agents and chemotherapeutic agents to be tested simultaneously against cells from samples of a subject's blood and/or tissue. In particular, the present inventors have devised an assay which (i) allows for the effect of immunotherapeutic agents to be directly assessed on circulating tumour cells (CTCs) and/or cancerous cells from a tumour biopsy; (ii) replicates the composition of the biopsy sample in a consistent manner, allowing the effects of different agents to be compared directly without variation in the biopsy material confounding the results; and/or (iii) compensates for low-levels of cancerous cells within a biopsy by incorporating CTCs from the subject's blood into test populations. The present invention therefore provides a significant improvement over conventional screening methods for personalised medicine, successfully identifying treatments in a high proportion (up to 90% of subjects) than conventional methods (approx. 55% success rate), and doing so more quickly (10 days compared with 45 days under current NHS timescales). This has the potential to offer significant benefits both in terms of reducing the need for hospital/clinic visits, increasing patient compliance, quality of life and clinical outcomes, as well as reducing care costs.

Accordingly, the invention provides a method of predicting responsiveness of a subject having cancer to one or more immunotherapeutic agent and one or more chemotherapeutic agent, said method comprising the steps of: (a) contacting at least one population of CD45⁺ cells and circulating tumour cells (CTCs) from said subject with at least one immunotherapeutic agent; (b) contacting at least one population of CTCs from said subject with at least one chemotherapeutic agent; and (c) determining the efficacy of the at least one immunotherapeutic agent and at least one therapeutic agent against the CTCs.

Each of the at least one immunotherapeutic agents may be contacted with a separate population of CD45⁺ cells and CTCs. Alternatively or in addition, each of the at least one chemotherapeutic agents may be contacted with a separate population of CTCs.

Said method may further comprise the steps of: (d) contacting at least one population of CD45⁺ cells, CTCs, cancer stem cells (CSCs) and tumour marker⁺ cells with at least one immunotherapeutic agent; and (e) determining the efficacy of the at least one immunotherapeutic agent against the CTCs, CSCs and/or tumour marker⁺ cells. Optionally the at least one immunotherapeutic agent may be the same at least one immunotherapeutic agent as used in step (a).

The method may further comprise the steps of: (f) contacting at least one population of CSCs and/or tumour marker⁺ cells with at least one chemotherapeutic agent; and (g) determining the efficacy of the at least one chemotherapeutic agent against the CSCs and/or tumour marker⁺ cells. Optionally: (i) the at least one population of CSCs and/or tumour marker⁺ cells further comprises CTCs, and the method further comprises determining the efficacy of the at least one chemotherapeutic agent against the CTCs; and/or (ii) the at least one chemotherapeutic agent is the same at least one chemotherapeutic agent as used in step (b).

The at least one population of: (i) CD45⁺ cells and CTCs; (ii) CTCs; (iii) CD45⁺ cells, CTCs, CSCs and tumour marker⁺ cells; and/or (iv) CSCs and/or tumour marker⁺ cells and optionally CTCs; may further comprise tumour marker-cells.

The CD45⁺ cells and/or CTCs may have been isolated from a blood sample from the subject. Alternatively or in addition, the CSCs, tumour marker⁺ cells and/or tumour marker cells may have been isolated from a sample of cancerous tissue from the subject.

The CD45⁺ cells, CTCs CSCs, tumour marker⁺ cells and/or tumour marker cells may be isolated from the sample using magnetic activated cell sorting (MACS), fluorescence activated cell sorting (FACS), flow cytometry, or buoyancy activated cell sorting (BACS).

The CD45⁺ cells may: (a) be haematopoietic in origin; and/or (b) originate from the bone marrow. The CD45⁺ cells may be lymphoid. The CD45⁺ cells and/or the CTCs are less than 20 μm in diameter.

The CTCs may be EpCAM⁺CD45⁻cells. The CSCs may be CD133⁺ cells. The tumour marker cells may be (i) CD31⁺, CD45⁺ and/or Gly-A⁺; and/or (ii) can be bound by an anti-fibroblast antibody. The tumour marker⁺ cells may be the remaining cells from the tissue sample that are not tumour marker-cells.

The CSCs, tumour marker⁺ cells and/or tumour marker cells may be present in essentially the same proportions as in the cancerous tissue sample.

The cancer may be a solid cancer. The solid cancer may be selected from lung cancer, breast cancer, colon cancer, prostate cancer, melanoma (skin cancer), kidney cancer (renal cell carcinoma), head and neck cancer (squamous cell carcinoma), pancreatic cancer, brain or CNS cancer, bladder cancer, oesophageal cancer, cancer of unknown primary, ovarian cancer, stomach cancer, liver cancer, thyroid cancer and uterine cancer.

All steps comprising contacting a cell population with at least one immunotherapeutic agent and/or at least one chemotherapeutic agent may be carried out simultaneously. The steps comprising contacting a cell population with at least one immunotherapeutic agent and/or at least one chemotherapeutic agent may be carried out sequentially.

Efficacy of the at least one immunotherapeutic agent and/or the at least one chemotherapeutic agent may be determined by cytotoxicity and/or cell viability assay. The at least one population of: (i) CD45⁺ cells and CTCs; (ii) CTCs; (iii) CD45⁺ cells, CTCs, CSCs and tumour marker⁺ cells; and/or (iv) CSCs and tumour marker⁺ cells and optionally CTCs; optionally further comprising tumour marker cells, may be plated onto a biocompatible scaffold prior to contacting with at least one immunotherapeutic agent and/or at least one chemotherapeutic agent. The biocompatible scaffold may comprise or consist of a layer of biocompatible electrospun polymer fibers. The electrospun polymer fibers may comprise a polymer selected from the group consisting of polyethylene terephthalate, silicone, polyurethane, polycarbonate, polyether ketone, polycaprolactone, polylactic acid, polyglycolic acid, collagen, gelatin, fibronectin, hyaluronic acid, and combinations thereof.

The time taken to predict responsiveness of a subject having cancer to one or more immunotherapeutic agent and one or more chemotherapeutic agent may be 15 days or less, preferably 10 days or less.

A method of the invention may have a success rate of at least 75%, preferably at least 80%, more preferably at least 90% of correctly predicting responsiveness of a subject having cancer to one or more immunotherapeutic agent and one or more chemotherapeutic agent.

The invention further provides a kit for use in the method of the invention, which comprises: (i) a biocompatible scaffold as defined herein, optionally attached to an inert support; (ii) means for isolating each of CD45⁺ cells, CTCs, CSCs, tumour marker⁺ cells and/or tumour marker cells; and/or (iii) one or more agent for determining cell viability and/or cytotoxicity. Said kit may optionally further comprise instructions for use.

The invention also provides a method of selecting a subject with cancer for treatment, said method comprising: (a) carrying out a method of the invention to predict the responsiveness of the subject to one or more immunotherapeutic agent and/or one or more chemotherapeutic agent; and (b) selecting the subject for treatment on the basis of their responsiveness to one or more immunotherapeutic agent and/or one or more chemotherapeutic agent.

The invention further provides a method of selecting an immunotherapeutic agent and/or chemotherapeutic agent for treating a subject with cancer, said method comprising: (a) carrying out a method of the invention to predict the responsiveness of the subject to one or more immunotherapeutic agent and/or one or more chemotherapeutic agent; and (b) selecting an immunotherapeutic agent and/or chemotherapeutic agent for treatment on the basis of the subject's responsiveness to the one or more immunotherapeutic agent and/or one or more chemotherapeutic agent.

The invention also provides a method of treating cancer in a subject in need thereof, said method comprising: (a) carrying out a method as defined herein to predict the responsiveness of the subject to one or more immunotherapeutic agent and/or one or more chemotherapeutic agent; and (b) administering an effective amount of the one or more immunotherapeutic agent and/or one or more chemotherapeutic agent to the subject.

### DETAILED DESCRIPTION OF THE INVENTION

The present disclosure is generally directed to methods of predicting the responsiveness of subjects to certain cancer treatments; for example, the methods may involve identification and/or selection of subjects as candidates for various cancer treatments. The disclosure may also provide methods of selecting a cancer treatment for a given subject, and methods of treating cancer. The disclosure may also provide methods of reducing the proliferation of cancer cells. The disclosure may also provide kits suitable for carrying out such methods.

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition.

It should be understood that this invention is not limited to the particular methodology, protocols, and reagents, etc., described herein and as such can vary. In particular, any methods and materials similar or equivalent to those described herein can be used in the practice or testing of embodiments of this disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The headings provided herein are not limitations of the various aspects or embodiments of this disclosure.

As used herein, the term “capable of when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The term “immunotherapeutic agent”, “immunotherapeutic” and “immunotherapy” are used interchangeably herein to refer to agents that stimulate or suppress the immune system to help the body fight cancer. Immunotherapy may only target certain cells of the immune system, or may affect the immune system in a general way. Non-limiting examples of immunotherapy include monoclonal antibodies, checkpoint inhibitors, vaccines, cytokines and CAR-T cell therapy.

The term “chemotherapeutic agent”, “chemotherapeutic” and “chemotherapy” are used interchangeably herein to refer to cytotoxic agents that kill cancer cells.

CD45 (lymphocyte common antigen) is a receptor-linked protein tyrosine phosphatase that is expressed on all leukocytes (white blood cells), and which plays a crucial role in the function of these cells. The term CD45⁻positive (CD45⁺) cells encompasses both CD45⁺ cells and CD45⁻high (CD45^{hi}) cells. Accordingly, CD45⁺ cells as defined herein are immune cells (or white blood cells) which play a role in the response of a subject's immune system to cancer. Typically CD45⁺ cells according to the invention are haematopoietic in origin, in other words they originate from the bone marrow. Typically CD45⁺ cells of the invention are lymphoid cells, i.e. natural killer cells, T lymphocytes or B lymphocytes, or a combination thereof.

The terms circulating tumour cells (CTCs), circulating cancer cells (CCCs) and circulating neoplastic cells (CNCs) are used interchangeably to refer to exfoliate neoplastic cells circulating in the blood. CTCs are associated with metastasising tumours. CTCs typically express epithelial cell adhesion molecule (EpCAM), i.e. are EpCAM-positive (EpCAM⁺) or EpCAM-high (EpCAM^{hi}) cells. Typically CTCs are CD45⁻negative (CD45) or CD45⁻low (CD45^{lo} ) cells. Preferably CTCs are EpCAM⁺CD45. Other markers that may be expressed by CTCs include cytokeratins, human epidermal growth factor receptor-2 (HER2), estrogen receptor, prostate-specific membrane antigen, folate receptor and/or survivin, depending on the cancer from which the CTCs are derived. Alternatively or in addition, CTCs may exhibit reproducible copy number alternations (CNAs) patterns, similar to those of metastatic tumours. CTCs are distinct from cancer associated macrophage-like cells (CAMLs). Thus, a reference to CTCs does not encompass CAMLs and a reference to CAMLs does not encompass CTCs.

The terms cancer stem cells (CSCs), tumour stem cells (TSCs) and neoplastic stem cells (NSCs) are used interchangeably to refer to highly proliferative, self-renewing, and colony-forming stem cells which give rise to neoplastic growth. CSCs express CD133, i.e. are CD133-positive (CD133⁺) or CD133⁻ high (CD133^{hi}) cells. Other markers that may be expressed by CSCs include CD44, ALDH1A1, CD34 and/or CD24. Typically CSCs have: (1) have extensive proliferative capacity; 2) are capable of asymmetric cell division to generate one or more types of differentiated cell progeny wherein the differentiated cells have reduced and/or limited proliferative or developmental potential; and (3) are capable of symmetric cell divisions for self-renewal or self-maintenance. These properties confer on the cancer stem cells the ability to form or establish a tumour or cancer upon serial transplantation into an immunocompromised host (e.g., a mouse) compared to the majority of tumour cells that fail to form tumours. Cancer stem cells undergo self-renewal versus differentiation in a chaotic manner to form tumours with abnormal cell types that can change over time as mutations occur.

The term “tumour marker negative cells” or “tumour marker cells” refers to cells which are positive or high for the expression of one or more of CD31, CD45 and/or Glycophorin-A (Gly-A) (i.e. CD31⁺, CD45⁺ and/or Gly-A⁺or CD31^{hi}, CD45^{hi} and/or Gly-A^{hi}), or any combination thereof. Preferably, tumour marker negative cells are positive for one or more of CD31, CD45 and/or Gly-A. Alternatively or in addition, a “tumour marker negative cell” or “tumour marker cell” may refer to a cell which can be bound or detected by an anti-fibroblast antibody. Such anti-fibroblast antibodies are well-known in the art, with many examples being commercially available, such as the anti-fibroblasts antibody, clone TE-7 (Sigma Aldrich) and the anti-fibroblast marker antibody ER-TR7 (Santa Cruz). Thus, the term “tumour marker negative cells” may encompass fibroblasts or fibroblast-like cells.

The term “tumour marker positive cells” or “tumour marker⁺ cells” as used herein refers to any cells other than tumour marker negative cells as defined herein. In other words, the term “tumour marker positive cells” or “tumour marker⁺ cells” as used herein refers to cells which are negative or low for the expression of one or more of CD31, CD45 and/or Glycophorin-A (Gly-A) (i.e. CD31⁻, CD45⁻and/or Gly-A⁻ or CD31^{lo} , CD45^{lo} and/or Gly-A^{lo}), or any combination thereof. Preferably, tumour marker positive cells are negative or low for expression of each of CD31, CD45 and Gly-A, most preferably negative for expression of each CD31, CD45 and Gly-A. Alternatively or in addition, a “tumour marker positive cell” or “tumour marker⁺ cell” may refer to a cell which cannot be bound or detected by an anti-fibroblast antibody (such as those described herein). Tumour marker positive cells may be defined and/or isolated from a tissue sample by identifying and/or isolating the tumour marker negative cells within a sample, such as by a method as described herein. All the remaining cells within the tissue sample (i.e. all cells not identified and/or isolated as tumour marker negative cells) may be defined as tumour marker positive cells according to the present invention.

Solid cancers may be defined as abnormal cellular growths in “solid” organs such as the breast or prostate, as opposed to leukaemia, a cancer affecting the blood, which is liquid.

Biocompatible materials, as defined herein are materials which are suitable for the culture of cells, particularly mammalian and preferably human cells. Biocompatible materials are typically suitable for use in medical applications within the body or on external surfaces, and elicit little or no immune response in human or veterinary applications. Thus, a biocompatible scaffold is a scaffold made from biocompatible material.

The term “scaffold” as used herein refers to a 2D or 3D scaffold, typically made of a polymer. The shape and dimensions of the scaffold may be based on a native biological structure, and are typically in microscale or nanoscale structures.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

It must be noted that as used herein and in the appended claims, the singular forms “a”, “an”, and “the” include plural referents unless the context clearly dictates otherwise. Thus, for example, reference to “a population” includes a plurality of populations and “an immunotherapeutic (or chemotherapeutic) agent” includes a plurality of such candidate agents. Similarly, reference to “the population” includes reference to one or more population and reference to “the immunotherapeutic (or chemotherapeutic) agent” includes reference to one or more such agent and equivalents thereof known to those skilled in the art, and so forth. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (+) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

An individual can be one who has been previously diagnosed with or identified as suffering from or having a condition in need of treatment or one or more complications related to such a condition, and optionally, have already undergone treatment for a condition as defined herein or the one or more complications related to said condition. Alternatively, an individual can also be one who has not been previously diagnosed as having a condition as defined herein or one or more complications related to said condition. For example, an individual can be one who exhibits one or more risk factors for a condition, or one or more complications related to said condition or a subject who does not exhibit risk factors.

An “individual in need” of treatment for a particular condition can be an individual having that condition, diagnosed as having that condition, or at risk of developing that condition.

The terms “subject”, “individual” and “patient” are used interchangeably herein to refer to a mammalian individual. An “individual” may be any mammal. Generally, the individual may be human; in other words, in one embodiment, the “individual” is a human. A “individual” may be an adult, juvenile or infant. An “individual” may be male or female.

The term “pharmaceutically acceptable” as used herein means approved by a regulatory agency of the Federal or a state government, or listed in the U.S. Pharmacopeia, European Pharmacopeia or other generally recognized pharmacopeia.

The publications discussed herein are provided solely for their disclosure prior to the filing date of the present application. Nothing herein is to be construed as an admission that such publications constitute prior art to the claims appended hereto. All references cited in this specification are herewith incorporated by reference with respect to their entire disclosure content and the disclosure content specifically mentioned in this specification.

### Screening for Responsiveness to Immunotherapeutic Agents

The invention provides a method of predicting responsiveness of a subject having cancer to one or more immunotherapeutic agent. Said method typically comprises the steps of contacting at least one population of CD45⁺ cells and circulating tumour cells (CTCs) from said subject with at least one immunotherapeutic agent and determining the efficacy of the at least one immunotherapeutic agent against the CTCs.

A population of CD45⁺ cells and CTCs typically comprises, consists essentially of, or consists of CD45⁺ cells and CTCs.

A plurality of populations of CD45⁺ cells and CTCs, e.g. at least two, at least three, at least four, at least five, at least six, at least seven, at least eight, at least nine, at least 10, or more populations may be contacted with an immunotherapeutic agent or combination thereof in a method of the invention.

A plurality of populations of CD45⁺ cells and CTCs may be from a single subject or from multiple (e.g. 2, 3, 4, 5, 6, 7, 8, 9, 10, 15, 20, 25 or more) subjects. Typically any individual population of CD45⁺ cells and CTCs within the plurality is from a single subject even when the plurality comprises populations from different subjects.

Each population of CD45⁺ cells and CTCs within a plurality of populations of CD45⁺ cells and CTCs may be contacted with a separate (i.e. different) immunotherapeutic agent or combination thereof. In other words, each immunotherapeutic agent or combination thereof may be used to contact a separate (i.e. different) population of CD45⁺ cells and CTCs within a plurality of CD45⁺ cells and CTCs populations. Alternatively, each immunotherapeutic agent or combination thereof may be used to contact multiple (e.g. 2, 3, 4 or more) populations of CD45⁺ cells and CTCs within a plurality of CD45⁺ cells and CTCs populations, e.g. in order to provide experimental replicates.

Wherein a plurality of populations of CD45⁺ cells and CTCs comprises populations of CD45⁺ cells and CTCs from multiple subjects, each immunotherapeutic agent or combination thereof may be used to contact at least one population of CD45⁺ cells and CTCs from each subject. In other words, at least one population of CD45⁺ cells and CTCs from each subject may be contacted with each immunotherapeutic agent or combination thereof.

The methods of the invention may be used to predict the responsiveness of a subject to any immunotherapeutic agent as defined herein, such as monoclonal antibodies, checkpoint inhibitors, vaccines, cytokines and CAR-T cell therapy, or a combination thereof. Non-limiting examples of immunotherapeutic agents include PD-1 inhibitors (such as pembrolizumab (Keytruda), nivolumab (Opdivo) and cemiplimab (Libtayo)),PD-L1 inhibitors (such as atezolizumab (Tecentriq), avelumab (Bavencio) and durvalumab (Imfinzi)) and/or CTLA-4 inhibitors (such as ipilimumab (Yervoy)).

The one or more immunotherapeutic agent may be tested at any appropriate concentration. The concentration(s) used may be selected based on the known range of concentrations used clinically. Thus, different immunotherapeutic agents may be tested at different concentrations. Selection of appropriate concentrations is within the routine practice of one of ordinary skill in the art. Typically the one or more immunotherapeutic agent is tested at a range of concentrations (e.g. at one, two, three, four, five, six, seven, eight, nine, ten or more different concentrations), to allow the sensitivity of the cancer cells within the sample to be determined.

The efficacy of an immunotherapeutic agent or combination thereof may be determined and/or quantified by any appropriate means. Suitable techniques are known in the art. Non-limiting examples include cytotoxicity assay, cell viability assay, fluorescent imaging, direct imaging and/or fluorescence measurement. In some preferred embodiments, efficacy of an immunotherapeutic or combination thereof is determined by cytotoxicity and/or cell viability. Cell viability relates to the quantification of the number of living cells. Cell viability can be quantified by imaging techniques (e.g. optical imagining or confocal imaging), metabolic assays (e.g. MTT assays or the RealTime-Glo™ MT assay from Promega (Catalogue No. G9711)), ATP content assays and/or cell proliferation assays, and may be colorimetric or fluorescence/luminescence-based. Preferably a metabolic assay is used according to the invention. Cytotoxicity assays measure parameters associated with loss of membrane integrity upon cell death. Non-limiting example of cytotoxicity assays that may be used according to the invention are the CellTox™ Green Cytotoxicity Assay (Promega, Catalogue No. G8741).

A subject may be predicted as responsive to an immunotherapeutic agent or combination thereof if the efficacy of said immunotherapeutic agent or combination thereof is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% efficacious against the CTCs within the one or more population of CD45⁺ cells and CTCs.

The efficacy of an immunotherapeutic agent or combination thereof may be quantified in absolute terms or relative to a control. Absolute quantification may be in terms of cell number or a readout for cell number (e.g. a colorimetric or fluorescent readout). Relative quantification is measured relative to a control. Thus, the efficacy (e.g. as measured using cell viability and/or cytotoxicity) of an immunotherapeutic agent or combination thereof against the CTCs within the one or more population of CD45⁺ cells and CTCs may be compared with the efficacy (e.g. as measured using cell viability and/or cytotoxicity) of an immunotherapeutic agent or combination thereof against a control. Typically the control is an equivalent population or sample in which no immunotherapeutic agent (or combination thereof) has been added, for example a population of CD45⁺ cells and CTCs obtained from the sample (or a different sample from the same subject) to which the immunotherapeutic agent (or combination thereof) has not been administered. Alternatively, a control may be a population of CTCs obtained from the sample (or a different sample from the same individual) without CD45⁺ cells. An alternative or additional control may be a population of CD45⁺ cells and CTCs obtained from a sample from an individual without cancer, or a healthy individual.

A subject may be predicted as responsive to an immunotherapeutic agent or combination thereof if said immunotherapeutic agent or combination thereof reduces the cell viability of the CTCs within the one or more population of CD45⁺ cells and CTCs by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% reduction in CTCs viability within the one or more population of CD45⁺ cells and CTCs. This reduction may be relative to a control, as described above.

Alternatively or in addition, a subject may be predicted as responsive to an immunotherapeutic agent or combination thereof if said immunotherapeutic agent or combination thereof results in an increase in cell death of the CTCs within the one or more population of CD45⁺ cells and CTCs by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% increase in cell death of the CTCs within the one or more population of CD45⁺ cells and CTCs.

The CD45⁺ cells and/or CTCs used in said method are typically isolated from a blood sample, with each subject providing one or more blood sample as described herein. Accordingly, a method of the invention may further comprise a step of isolating CD45⁺ cells and/or CTCs from a sample. Any appropriate technique may be used to isolate the CD45⁺ cells and/or CTCs from the sample. Such techniques are well-known in the art. Non-limiting examples of suitable techniques include magnetic activated cell sorting (MACS), fluorescence activated cell sorting (FACS), flow cytometry, and buoyancy activated cell sorting (BACS). In some preferred embodiments, MACS is used to isolate CD45⁺ cells and/or CTCs from a sample. By way of non-limiting example, MACS may be used with beads having a CD45 binding agent (e.g. an anti-CD45 antibody) to isolate CD45⁺ cells. By way of a further non-limiting example, beads with an EpCAM binding agent (e.g. an anti-EpCAM antibody) may be used to isolate CTCs via MACS. Isolation techniques may exclude or prevent the isolation of cells of above a certain size or diameter. By way of non-limiting example, when MACS is used to isolate CD45⁺ cells and/or CTCs from a sample as described herein, the size of tubes used for MACS may restricts some types of cells from being isolated due to their larger size. In some preferred embodiments, the use of MACS to isolate CD45⁺ cells and/or CTCs according to the invention may exclude the isolation of CAMLs with the CD45⁺ cells and/or CTCs.

CD45⁺ cells used in a method of the invention are typically haematopoietic in origin, in other words they originate from the bone marrow. Preferably, CD45⁺ cells of the invention are lymphoid cells. Thus, CD45⁺ cells used in a method of the invention may be (i) T lymphocytes; (ii) B lymphocytes; (iii) natural killer cells; (iv) common lymphoid progenitor cells or any combination of (i) to (iv). Preferably, CD45⁺ cells used in a method of the invention may be (i) T lymphocytes; (ii) B lymphocytes; (iii) natural killer cells; or any combination of (i) to (iii). More preferably the CD45⁺ cells used in a method of the invention are T lymphocytes, B lymphocytes or a combination of T and B lymphocytes, optionally further comprising natural killer cells. CD45⁺ cells used in a method of the invention may be less than about 20 μm in diameter, such as less than about 20 μm in diameter, less than about 19 μm in diameter, less than about 18 μm in diameter, less than about 17 μm in diameter, less than about 16 μm in diameter, less than about 15 μm in diameter or less. Preferably CD45⁺ cells used in a method of the invention are less than 20 μm in diameter.

Alternatively or in addition, CTCs used in a method of the invention may be less than about 20 μm in diameter, such as less than about 20 μm in diameter, less than about 19 μm in diameter, less than about 18 μm in diameter, less than about 17 μm in diameter, less than about 16 μm in diameter, less than about 15 μm in diameter, less than about 14 μm in diameter, less than about 13 μm in diameter, less than about 12 μm in diameter, less than about 11 μm in diameter, less than about 10 μm in diameter or less. Preferably CTCs used in a method of the invention are less than 20 μm in diameter. In some embodiments, the average diameter of CTCs used in methods of the invention is between about 10 μm to about 20 μm, such as between about 10 μm to about 15 μm or between about 12 μm to about 15 μm. In some embodiments, the average diameter of CTCs used in methods of the invention is about 12.4 μm.

In some preferred embodiments, CD45⁺ cells used in a method of the invention may be less than 20 μm in diameter and CTCs used in a method of the invention may be less than 20 μm in diameter.

Size of the CD45⁺ cells and/or CTCs may be determined using any appropriate technique, examples of which are known in the art, such as flow cytometry.

In embodiments where a subject further provides a tissue sample (e.g. a sample comprising or consisting of cancerous tissue, such as a tissue biopsy), a method of the invention may further comprise the steps of: contacting at least one population of (i) CD45⁺ cells, (ii) CTCs and (iii) cancer stem cells (CSCs) and/or tumour marker⁺ cells; with at least one immunotherapeutic agent or combination thereof; and determining the efficacy of the at least one immunotherapeutic agent or combination thereof against the CTCs, CSCs and/or tumour marker⁺ cells.

A population of (i) CD45⁺ cells, (ii) CTCs and (iii) cancer stem cells (CSCs) and/or tumour marker⁺ cells typically comprises, consists essentially of, or consist of (i) CD45⁺ cells, (ii) CTCs and (iii) cancer stem cells (CSCs) and/or tumour marker⁺ cell.

A plurality of populations of (i) CD45⁺ cells, (ii) CTCs and (iii) cancer stem cells (CSCs) and/or tumour marker⁺ cells, e.g. at least two, at least three, at least four, at least five, at least six, at least seven, at least eight, at least nine, at least 10, or more populations may be contacted with an immunotherapeutic agent or combination thereof in a method of the invention.

A plurality of populations of (i) CD45⁺ cells, (ii) CTCs and (iii) cancer stem cells (CSCs) and/or tumour marker⁺ cells may be from a single subject or from multiple (e.g. 2, 3, 4, 5, 6, 7, 8, 9, 10, 15, 20, 25 or more) subjects. Typically any individual population of (i) CD45⁺ cells, (ii) CTCs and (iii) cancer stem cells (CSCs) and/or tumour marker⁺ cells within the plurality is from a single subject, even when the plurality comprises populations from different subjects.

Each population of (i) CD45⁺ cells, (ii) CTCs and (iii) cancer stem cells (CSCs) and/or tumour marker⁺ cells within a plurality of populations of (i) CD45⁺ cells, (ii) CTCs and (iii) cancer stem cells (CSCs) and/or tumour marker⁺ cells may be contacted with a separate (i.e. different) immunotherapeutic agent or combination thereof. In other words, each immunotherapeutic agent or combination thereof may be used to contact a separate (i.e. different) population of (i) CD45⁺ cells, (ii) CTCs and (iii) cancer stem cells (CSCs) and/or tumour marker⁺ cells within a plurality of (i) CD45⁺ cells, (ii) CTCs and (iii) cancer stem cells (CSCs) and/or tumour marker⁺ cells populations. Alternatively, each immunotherapeutic agent or combination thereof may be used to contact multiple (e.g. 2, 3, 4 or more) populations of (i) CD45⁺ cells, (ii) CTCs and (iii) cancer stem cells (CSCs) and/or tumour marker⁺ cells within a plurality of (i) CD45⁺ cells, (ii) CTCs and (iii) cancer stem cells (CSCs) and/or tumour marker⁺ cells, e.g. in order to provide experimental replicates.

Wherein a plurality of populations of (i) CD45⁺ cells, (ii) CTCs and (iii) cancer stem cells (CSCs) and/or tumour marker⁺ cells comprises populations of (i) CD45⁺ cells, (ii) CTCs and (iii) cancer stem cells (CSCs) and/or tumour marker⁺ cells from multiple subjects, each immunotherapeutic agent or combination thereof may be used to contact at least one population of (i) CD45⁺ cells, (ii) CTCs and (iii) cancer stem cells (CSCs) and/or tumour marker⁺ cells from each subject. In other words, at least one population of (i) CD45⁺ cells, (ii) CTCs and (iii) cancer stem cells (CSCs) and/or tumour marker⁺ cells from each subject may be contacted with each immunotherapeutic agent or combination thereof.

The methods of the invention may be used to predict the responsiveness of a subject to any immunotherapeutic agent as defined herein using at least one population of (i) CD45⁺ cells, (ii) CTCs and (iii) cancer stem cells (CSCs) and/or tumour marker⁺ cells. The disclosure herein in relation immunotherapeutic agents and combinations thereof which can be tested according to the present invention in the context of CD45⁺ cells and CTCs applies equally and without reservation to testing at least one population of (i) CD45⁺ cells, (ii) CTCs and (iii) cancer stem cells (CSCs) and/or tumour marker⁺ cells. In other words, the immunotherapeutic agents and combinations thereof as described herein can also be tested on at least one population of (i) CD45⁺ cells, (ii) CTCs and (iii) cancer stem cells (CSCs) and/or tumour marker⁺ cells. In some embodiments, the at least one immunotherapeutic agent and combinations thereof used to contact the at least one population of (i) CD45⁺ cells, (ii) CTCs and (iii) cancer stem cells (CSCs) and/or tumour marker⁺ cells is the same as the at least one immunotherapeutic agent or combination thereof used to contact the at least one population of CD45⁺ cells and CTCs.

The efficacy of an immunotherapeutic agent or combination thereof against a population of (i) CD45⁺ cells, (ii) CTCs and (iii) cancer stem cells (CSCs) and/or tumour marker⁺ cells may be determined and/or quantified by any appropriate means, as described above.

A subject may be predicted as responsive to an immunotherapeutic agent or combination thereof if the efficacy of said immunotherapeutic agent or combination thereof is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% efficacious against the CTCs, CSCs and/or tumour marker⁺ cells within the one or more population of (i) CD45⁺ cells, (ii) CTCs and (iii) cancer stem cells (CSCs) and/or tumour marker⁺ cells.

The efficacy of an immunotherapeutic agent or combination thereof may be quantified in absolute terms or relative to a control, as described above. Thus, the efficacy (e.g. as measured using cell viability and/or cytotoxicity) of an immunotherapeutic agent or combination thereof against the CTCs, CSCs and/or tumour marker⁺ cells within the one or more population of (i) CD45⁺ cells, (ii) CTCs and (iii) cancer stem cells (CSCs) and/or tumour marker⁺ cells may be compared with the efficacy (e.g. as measured using cell viability and/or cytotoxicity) of an immunotherapeutic agent or combination thereof against a control. Typically the control is an equivalent population or sample in which no immunotherapeutic agent (or combination thereof) has been added, for example a population of (i) CD45⁺ cells, (ii) CTCs and (iii) cancer stem cells (CSCs) and/or tumour marker⁺ cells obtained from the sample (or a different sample from the same subject) to which the immunotherapeutic agent (or combination thereof) has not been administered. Alternatively, a control may be a population of CTCs, CSCs and/or tumour marker⁺ cells obtained from the sample (or a different sample from the same individual) without CD45⁺ cells. An alternative or additional control may be a population of (i) CD45⁺ cells, (ii) CTCs and (iii) cancer stem cells (CSCs) and/or tumour marker⁺ cells obtained from a sample from an individual without cancer, or a healthy individual.

A subject may be predicted as responsive to an immunotherapeutic agent or combination thereof if said immunotherapeutic agent or combination thereof reduces the cell viability of the CTCs, CSCs and/or tumour marker⁺ cells within the one or more population of (i) CD45⁺ cells, (ii) CTCs and (iii) cancer stem cells (CSCs) and/or tumour marker⁺ cells by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% reduction in CTCs, CSCs and/or tumour marker⁺ cell viability within the one or more population of (i) CD45⁺ cells, (ii) CTCs and (iii) cancer stem cells (CSCs) and/or tumour marker⁺ cells. This reduction may be relative to a control, as described above.

Alternatively or in addition, a subject may be predicted as responsive to an immunotherapeutic agent or combination thereof if said immunotherapeutic agent or combination thereof results in an increase in cell death of the CTCs, CSCs and/or tumour marker⁺ cells within the one or more population of (i) CD45⁺ cells, (ii) CTCs and (iii) cancer stem cells (CSCs) and/or tumour marker⁺ cells by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% increase in cell death of the CTCs, CSCs and/or tumour marker⁺ cells within the one or more population of (i) CD45⁺ cells, (ii) CTCs and (iii) cancer stem cells (CSCs) and/or tumour marker⁺ cells.

The CSCs and/or tumour marker⁺ cells used in said method are typically isolated from a tissue sample (e.g. a sample comprising or consisting of cancerous tissue, such as a tissue biopsy), with each subject providing one or more tissue sample as described herein. Accordingly, a method of the invention may further comprise a step of isolating CSCs and/or tumour marker⁺ cells from a sample. Any appropriate technique may be used to isolate the CSCs and/or tumour marker⁺ cells from the sample. Such techniques are well-known in the art. Non-limiting examples of suitable techniques include magnetic activated cell sorting (MACS), fluorescence activated cell sorting (FACS), flow cytometry, and buoyancy activated cell sorting (BACS). In some preferred embodiments, MACS is used to isolate CSCs and/or tumour marker⁺ cells from a sample. By way of non-limiting example, MACS may be used with beads having a CD133 binding agent (e.g. an anti-CD133 antibody) to isolate CD133⁺ cells (CSCs). By way of a further non-limiting example, beads with at least one binding agent for one or more of CD31, CD45 and/or Gly-A (e.g. anti-CD31, anti-CD45 and/or anti-Gly-A antibodies) and/or an anti-fibroblast antibody as described herein may be used to isolate tumour marker cells via MACS. Having isolated tumour marker cells (e.g. using MACS), the remaining cells in a tissue sample may be defined as tumour marker⁺ cells.

The CSCs and/or tumour marker⁺ cells, preferably both the CSCs and tumour marker⁺ cells when present within populations to be contacted with the at least one immunotherapeutic agent may be present in essentially the same proportions as the CSCs and/or tumour marker⁺ cells are present in the tissue sample. By “essentially the same proportions” it is typically meant that the proportions of CSCs and/or tumour marker⁺ cells are the same as the proportions within the tissue sample, within the limits of experimental error and/or accounting for cell loss during sample processing/isolation of the cells. By way of non-limiting example, if CSCs and tumour marker⁺ cells are present at a ratio of 1:10 within the tissue sample, then populations comprising CSCs and tumour marker⁺ cells for use in methods of the invention may comprise CSCs and tumour marker⁺ cells at a ratio of about 1:10 (e.g. from about 1:8 to about 1:12). Using populations in which CSCs and/or tumour marker⁺ cells are present in essentially the same proportions as the CSCs and/or tumour marker⁺ cells are present in the tissue sample advantageously allows for the effects of the at least one immunotherapeutic agent on the CSCs and/or tumour marker⁺ cells to be investigated in an environment that replicates in vitro the environment present within the tissue sample, and hence the subject's tumour or cancer. Populations comprising essentially the same proportions of CSCs and/or tumour marker⁺ cells may be obtained from a tissue sample by isolating the CSCs and/or tumour marker⁺ cells, and dividing the isolated CSCs and/or tumour marker⁺ cells equally into populations. The number of CSCs and/or tumour marker⁺ cells may be counted prior to dividing into populations, if a certain number of CSCs and/or tumour marker⁺ cells is required per population. Typically by dividing the CSCs and/or tumour marker⁺ cells obtained from a tissue sample into equal populations, and using these populations for each test condition and control, the proportion of CSCs and/or tumour marker⁺ cells used for each test condition and control will be essentially the same as each other and essentially the same as the proportion of CSCs and/or tumour⁺ cells within the tissue sample, and hence the subject's tumour or cancer. By way of quality control, the number of different cell types (e.g. CSCs and/or tumour marker⁺ cells) may be counted to ensure that the sample can be processed according to the invention. Alternatively or in addition, the number of populations may depend on the number of immunotherapeutic agents to be tested.

In a similar way, CD45⁺ cells and/or CTCs, preferably both the CD45⁺ cells and CTCs when present within populations to be contacted with the at least one immunotherapeutic agent may be present in essentially the same proportions as the CD45⁺ cells and/or CTCs are present in the blood sample. By “essentially the same proportions” it is typically meant that the proportions of CD45⁺ cells and/or CTCs cells are the same as the proportions within the blood sample, within the limits of experimental error and/or accounting for cell loss during sample processing/isolation of the cells. By way of non-limiting example, if CD45⁺ cells and CTCs are present at a ratio of 10:1 within the blood sample, then populations comprising CD45⁺ cells and CTCs for use in methods of the invention may comprise CD45⁺ cells and CTCs at a ratio of about 10:1 (e.g. from about 8:1 to about 12:1). Using populations in which CD45⁺ cells and/or CTCs are present in essentially the same proportions as the CD45⁺ cells and/or CTCs are present in the blood sample advantageously allows for the effects of the at least one immunotherapeutic agent on the CD45⁺ cells and/or CTCs to be investigated in an environment that replicates in vitro the environment present within the blood sample, and hence the subject. Populations comprising essentially the same proportions of CD45⁺ cells and/or CTCs may be obtained from a blood sample by isolating the CD45⁺ cells and/or CTCs, and dividing the isolated CD45⁺ cells and/or CTCs cells equally into populations. The number of CD45⁺ cells and/or CTCs may be counted prior to dividing into populations, if a certain number of CD45⁺ cells and/or CTCs is required per population. Typically by dividing the CD45⁺ cells and/or CTCs obtained from a blood sample into equal populations, and using these populations for each test condition and control, the proportion of CD45⁺ cells and/or CTCs used for each test condition and control will be essentially the same as each other and essentially the same as the proportion of CD45⁺ cells and/or CTCs within the blood sample, and hence the subject. By way of quality control, the number of different cell types (e.g. CD45⁺ cells and/or CTCs) may be counted to ensure that the sample can be processed according to the invention. Alternatively or in addition, the number of populations may depend on the number of immunotherapeutic agents to be tested.

All steps comprising contacting a cell population with at least one immunotherapeutic agent may be carried out simultaneously in a method of the invention. Simultaneous contacting of populations with at least one immunotherapeutic agent may be desirable, for example, when all populations are being contacted with the same at least one immunotherapeutic, or when different immunotherapeutic agents are used but each requires the same incubation time. Alternatively, the steps comprising contacting a cell population with at least one immunotherapeutic agent may be carried out sequentially. Sequential contracting of populations with at least one immunotherapeutic may be desirable, for example, when different immunotherapeutic agents are being used, and one or more requires a different incubation time. In some embodiments, two or more populations may be contacted with at least one immunotherapeutic agent simultaneously, with one or more additional population being contacted with at least one immunotherapeutic agent (typically a different immunotherapeutic agent) sequentially.

A method of the invention may reduce the time taken to predict responsiveness of a subject to one or more immunotherapeutic agent. By way of non-limiting example, a method of the invention may reduce the time taken to predict responsiveness to one or more immunotherapeutic agent by at least 1 day, at least 2 days, at least 3 days, at least 4 days, at least 5 days, at least 6 days, at least 7 days, at least 8 days, at least 9 days, at least 10 days, at least 2 weeks, at least 3 weeks, at least 4 weeks or more. Typically such a reduction in time is compared with suitable control, such as a conventional screening method, including those used in current standards of care (e.g. within the NHS).

A method of the invention may reduce the time taken to predict responsiveness of a subject to one or more immunotherapeutic agent. By way of non-limiting example, the time taken to predict responsiveness to one or more immunotherapeutic agent using a method of the invention may be 20 days or less, 15 days or less, 14 days or less, 13 days or less, 12 days or less, 11 days or less, or 10 days or less. Preferably the time taken to predict responsiveness to one or more immunotherapeutic agent using a method of the invention is 15 days or less more preferably 10 days or less. The time taken to predict responsiveness to one or more immunotherapeutic agent using a method of the invention may be between about 4 to about 7 days, such as about 5 days.

A method of the invention may increase the success rate of correctly predicting responsiveness of a subject to one or more immunotherapeutic agent. By way of non-limiting example, a method of the invention may increase the success rate 10%, at least 20%, at least 30%, at least 40% at least 50% at least 60%, at least 70%, at least 80%, or more. Typically such an increase in the success rate is compared with suitable control, such as a conventional screening method, including those used in current standards of care (e.g. within the NHS).

A method of the invention may have a success rate of correctly predicting responsiveness of a subject to one or more immunotherapeutic agent of at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more. Preferably the success rate of a method of the invention is at least 75%, more preferably at least 80%, even more preferably at least 90%.

A method of the invention typically allows for predicting responsiveness of a subject having cancer to one or more immunotherapeutic agent, by directly assessing the activity of CD45⁺ cells from said subject against CTCs and/or CSCs from said subject in the presence of one or more immunotherapeutic agent. Said method may comprise the steps of investigating the activity of at least one population of CD45⁺ cells against CTCs and/or CSCs from said subject contacted with at least one immunotherapeutic agent and determining the efficacy of the at least one immunotherapeutic agent against the CTCs and/or CSCs. Thus, the methods of the invention focus on the activity of the CD45⁺ cells against CTCs and/or CSCs, rather than physical properties (e.g. size or diameter) or other characteristics of these cell types.

### Screening for Responsiveness to Chemotherapeutic Agents

The invention provides a method of predicting responsiveness of a subject having cancer to one or more chemotherapeutic agent. Said method typically comprises the steps of contacting at least one population of CTCs from said subject with at least one chemotherapeutic agent and determining the efficacy of the at least one chemotherapeutic agent against the CTCs. The use of CTCs in a method of predicting responsiveness of a subject having cancer to one or more chemotherapeutic agent is typical when the method is carried out using a blood sample from said subject.

A population of CTCs typically comprises, consists essentially of, or consists of CTCs, as defined herein.

A plurality of populations of CTCs, e.g. at least two, at least three, at least four, at least five, at least six, at least seven, at least eight, at least nine, at least 10, or more populations may be contacted with an chemotherapeutic agent or combination thereof in a method of the invention.

A plurality of populations of CTCs may be from a single subject or from multiple (e.g. 2, 3, 4, 5, 6, 7, 8, 9, 10, 15, 20, 25 or more) subjects. Typically any individual population of CTCs within the plurality is from a single subject, even when the plurality comprises populations from different subjects.

Each population of CTCs within a plurality of populations of CTCs may be contacted with a separate (i.e. different) chemotherapeutic agent or combination thereof. In other words, each chemotherapeutic agent or combination thereof may be used to contact a separate (i.e. different) population of CTCs within a plurality of CTC populations. Alternatively, each chemotherapeutic agent or combination thereof may be used to contact multiple (e.g. 2, 3, 4 or more) populations of CTCs within a plurality of CTC populations, e.g. in order to provide experimental replicates.

Wherein a plurality of populations of CTCs comprises populations of CTCs from multiple subjects, each chemotherapeutic agent or combination thereof may be used to contact at least one population of CTCs from each subject. In other words, at least one population of CTCs from each subject may be contacted with each chemotherapeutic agent or combination thereof.

The invention provides a method of predicting responsiveness of a subject having cancer to one or more chemotherapeutic agent, said method comprising the steps of contacting at least one population of CSCs and/or tumour marker⁺ cells from said subject with at least one chemotherapeutic agent and determining the efficacy of the at least one chemotherapeutic agent against the CSCs and/or tumour marker⁺ cells. The use of CSCs and/or tumour marker⁺ cells in a method of predicting responsiveness of a subject having cancer to one or more chemotherapeutic agent is typical when the method is carried out using a tissue sample from said subject.

A population of CSCs typically comprises, consists essentially of, or consists of CSCs. A population of tumour marker⁺ cells typically comprises, consists essentially of, or consists of tumour marker⁺ cells. A population may comprise, consists essentially of, or consists of CSCs and tumour marker⁺ cells.

A plurality of populations of CSCs and/or tumour marker⁺ cells, e.g. at least two, at least three, at least four, at least five, at least six, at least seven, at least eight, at least nine, at least 10, or more populations may be contacted with an chemotherapeutic agent or combination thereof in a method of the invention.

A plurality of populations of CSCs and/or tumour marker⁺ cells may be from a single subject or from multiple (e.g. 2, 3, 4, 5, 6, 7, 8, 9, 10, 15, 20, 25 or more) subjects. Typically any individual population of CSCs and/or tumour marker⁺ cells within the plurality is from a single subject, even when the plurality comprises populations from different subjects.

Each population of CSCs and/or tumour marker⁺ cells within a plurality of populations of CSCs and/or tumour marker⁺ cells may be contacted with a separate (i.e. different) chemotherapeutic agent or combination thereof. In other words, each chemotherapeutic agent or combination thereof may be used to contact a separate (i.e. different) population of CSCs and/or tumour marker⁺ cells within a plurality of CSCs and/or tumour marker⁺ cells populations. Alternatively, each chemotherapeutic agent or combination thereof may be used to contact multiple (e.g. 2, 3, 4 or more) populations of CSCs and/or tumour marker⁺ cells within a plurality of CSCs and/or tumour marker⁺ cells populations, e.g. in order to provide experimental replicates.

Wherein a plurality of populations of CSCs and/or tumour marker⁺ cells comprises populations of CSCs and/or tumour marker⁺ cells from multiple subjects, each chemotherapeutic agent or combination thereof may be used to contact at least one population of CSCs and/or tumour marker⁺ cells from each subject. In other words, at least one population of CSCs and/or tumour marker⁺ cells from each subject may be contacted with each chemotherapeutic agent or combination thereof.

The methods of the invention may be used to predict the responsiveness of a subject to any chemotherapeutic agent as defined herein, such as alkylating agents, antimetabolites, plant alkaloids, mitotic inhibitors, antitumour antibiotics, platinum-based chemotherapeutics and topoisomerase inhibitors. Non-limiting examples of chemotherapeutics include: nitrogen mustards, ethylenimine derivatives, alkyl sulfonates, nitrosoureas and triazenes) such as uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexylen®, Hexastat®), triethylenethiophosphoramine, Temozolomide (Temodar®), thiotepa (Thioplex®), busulfan (Busilvex®, Myleran®), carmustine (BICNU®), lomustine (CeeNU®), streptozocin (Zanosar®), and Dacarbazine (DTIC-Dome®); folic acid antagonists (also referred to herein as antifolates), pyrimidine analogs, purine analogs and adenosine deaminase inhibitors) such as methotrexate (Rheumatrex®, Trexall®), 5-fluorouracil (Adrucil®, Efudex®, Fluoroplex®), floxuridine (FUDF®), cytarabine (Cytosar-UR, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), mercaptopurine (Puri-Nethol®), capecitabine (Xeloda®), nelarabine (Arranon®), azacitidine (Vidaza®) and gemcitabine (Gemzar®). Preferred antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®); vinca alkaloids such as vinblastine (Velban®, Velsar®), vincristine (Vincasar®, Oncovin®), vindesine (Eldisine®), vinorelbine (Navelbine®); carboplatin (Paraplat®, Paraplatin®), cisplatin (Platinol®), oxaliplatin (Eloxatin®); anthracyclines such as daunorubicin (Cerubidine®, Rubidomycin®), doxorubicin (Adriamycin®), epirubicin (Ellence®), idarubicin (Idamycin®), mitoxantrone (Novantrone®), valrubicin (Valstar®). Preferred anthracyclines include daunorubicin (Cerubidine®, Rubidomycin®) and doxorubicin (Adriamycin®); topotecan (Hycamtin®), irinotecan (Camptosar®), etoposide (Toposar®, VePesid®), teniposide (Vumon®), lamellarin D, SN-38, camptothecin (e.g., IT-101); taxanes such as paclitaxel (Taxol®), docetaxel (Taxotere®), larotaxel, cabazitaxel; epothilones such as ixabepilone, epothilone B, epothilone D, BMS310705, dehydelone, ZK-Epothilone (ZK-EPO); actinomycin (Cosmegen®), bleomycin (Blenoxane®), hydroxyurea (Droxia®, Hydrea®), mitomycin (Mitozytrex®, Mutamycin®); bruton's tyrosine kinase inhibitors which include without limitation terreic acid; calcineurin inhibitors which include without limitation cypermethrin, deltamethrin, fenvalerate and tyrphostin 8; CaM kinase II inhibitors which include without limitation 5-Isoquinolinesulfonic acid, 4- [{2S)-2-[(5-isoquinolinylsulfonyl)methylamino]-3-oxo-3-{4-phenyl-|-pipe-razinyl) propyl]phenyl ester and benzenesulfonamide; CD45 tyrosine phosphatase inhibitors which include without limitation phosphonic acid; CDC25 phosphatase inhibitors which include without limitation 1,4-naphthalene dione, 2,3-bis[(2-hydroxyethyl)thio]-(9Cl); CHK kinase inhibitors which include without limitation debromohymenialdisine; cyclooxygenase inhibitors which include without limitation 1H-indole-3-acetamide, I-(4-chlorobenzoyl)-5-methoxy-2-methyl-N-(2-phenylethyl)-(9Cl), 5-alkyl substituted 2-arylaminophenylacetic acid and its derivatives (e.g., celecoxib (Celebrex®), rofecoxib (Vioxx®), etoricoxib (Arcoxia®), lumiracoxib (Prexige®), valdecoxib (Bextra®) or 5-alkyl-2-arylaminophenylacetic acid); CRAF kinase inhibitors which include without limitation 3-(3,5-dibromo-4-hydroxybenzylidene)-5-iodo-1,3-dihydroindol-2-one and benzamide, 3-(dimethylamino)-N-[3-[(4-hydroxybenzoyl)amino]-4-methylphenyl]-(9Cl); cyclin dependent kinase inhibitors which include without limitation olomoucine and its derivatives, purvalanol B, roascovitine (Seliciclib®), indirubin, kenpaullone, purvalanol A and indirubin-3′-monooxime; cysteine protease inhibitors which include without limitation 4-morpholinecarboxamide, N-[IS)-3-fluoro-2-oxo-I-(2-phenylethyl) propyl]amino]-2-oxo-I-(phenylmethy-I)ethyl]-(9Cl); DNA intercalators which include without limitation plicamycin (Mithracin®) and daptomycin (Cubicin®); DNA strand breakers which include without limitation bleomycin (Blenoxane®). E3 ligase inhibitors which include without limitation N-((3,3,3-trifruoro-2-trifluoromethyl) propionyl) sulfanilamide; histone deacetylase (HDAC) inhibitors which include without limitation suberoylanilide hydroxamic acid (SAHA), [4-(2-amino-phenylcarbamoyl)-benzyl]-carbamic acid pyridine-3-ylmethylester and its derivatives, butyric acid, pyroxamide, trichostatin A, oxamflatin, apicidin, depsipeptide, depudecin, trapoxin and compounds disclosed in WO 02/22577; I-kappa B-alpha kinase inhibitors (IKK) which include without limitation 2-propenenitrile, 3-[(4-methylphenyl) sulfonyl]-(2E)-(9Cl); imidazotetrazinones which include without limitation temozolomide (Methazolastone®, Temodar® and its derivatives (e.g., as disclosed generically and specifically in U.S. Pat. No. 5,260,291) and Mitozolomide; insulin tyrosine kinase inhibitors which include without limitation hydroxyl-2-naphthalenylmethylphosphonic acid; c-Jun-N-terminal kinase (JNK) inhibitors which include without limitation pyrazoleanthrone and epigallocatechin gallate; mitogen-activated protein kinase (MAP) inhibitors which include without limitation benzenesulfonamide, N-[2-[3-(4-chlorophenyl)-2-propenyl]methyl]amino]methyl]phenyl]-N-(2-hy-droxyethyl)-4-methoxy-(9Cl); MDM2 inhibitors which include without limitation trans-4-iodo, 4′-boranyl-chalcone. MEK inhibitors which without limitation include butanedinitrile, bis[amino[2-aminophenyl)thio]methylene]-(9Cl); MMP inhibitors which include without limitation Actinonin, epigallocatechin gallate, collagen peptidomimetic and non-peptidomimetic inhibitors, tetracycline derivatives marimastat (Marimastat®), prinomastat, incyclinide (Metastat®), shark cartilage extract AE-941 (eovastat®), Tanomastat, TAA21 1, MMI270B or AAJ996; mTor inhibitors which include without limitation rapamycin (Rapamune®), and analogs and derivatives thereof, AP23573 (also known as ridaforolimus, deforolimus, or MK-8669), CCI-779 (also known as temsirolimus) (Torisel®) and SDZ-RAD; NGFR tyrosine kinase inhibitors which include without limitation tyrphostin AG 879; p38 MAP kinase inhibitors which include without limitation Phenol, 4-[4-(4-fluorophenyl)-5-(4-pyridinyl)-IH-imidazol-2-yl]-(9Cl), and benzamide, 3-(dimethylamino)-N-[3-[(4-hydroxylbenzoyl)amino]-4-methylphenyl]-(9Cl); p56 tyrosine kinase inhibitors which include without limitation damnacanthal and tyrphostin 46; PDGF pathway inhibitors which include without limitation tyrphostin AG 1296, tyrphostin 9,1,3-butadiene-1,1,3-tricarbonitrile, 2-amino-4-(IH-indol-5-yl)-(9Cl), imatinib (Gleevec®) and gefitinib (Iressa®); phosphatidylinositol 3-kinase inhibitors which include without limitation wortmannin, and quercetin dihydrate; phosphatase inhibitors which include without limitation cantharidic acid, cantharidin, and L-leucinamide; protein phosphatase inhibitors which include without limitation cantharidic acid, cantharidin, L-P-bromotetramisole oxalate, 2 (5H)-furanone, 4-hydroxy-5-(hydroxymethyl)-3-(I-oxohexadecyl)-(5R)-(9Cl) and benzylphosphonic acid; PKC inhibitors which include without limitation I-H-pyrollo-2,5-dione,3-I-[3-(dimethylamino) propyl]-IH-indol-3-yl]-4-(IH--indol-3-yl)-(9Cl), Bisindolylmaleimide IX, Sphinogosine, staurosporine, and Hypericin; PKC delta kinase inhibitors which include without limitation rottlerin; polyamine synthesis inhibitors which include without limitation DMFO; proteasome inhibitors which include, without limitation aclacinomycin A, gliotoxin and bortezomib (Velcade®). PTP1B inhibitors which include without limitation L-leucinamide. protein tyrosine kinase inhibitors which include, without limitation tyrphostin Ag 216, tyrphostin Ag 1288, tyrphostin Ag 1295, geldanamycin, genistein and 7H-pyrollo[2,3-d]pyrimidine derivatives as generically and specifically described in PCT Publication No. WO 03/013541 and U.S. Publication No. 2008/0139587.

SRC family tyrosine kinase inhibitors which include without limitation PP 1 and PP2.

Syk tyrosine kinase inhibitors which include without limitation piceatannol: Janus (JAK-2 and/or JAK-3) tyrosine kinase inhibitors which include without limitation tyrphostin AG 490 and 2-naphthyl vinyl ketone; retinoids which include without limitation isotretinoin (Accutane®, Amnesteem®, Cistane®, Claravis®, Sotret®) and tretinoin (Aberel®, Aknoten®, Avita®, Renova®, Retin-AR, Retin-A MICRO®, Vesanoid®); RNA polymerase II elongation inhibitors which include without limitation 5,6-dichloro-1-beta-D-ribofuranosylbenzimidazol; serine/Threonine kinase inhibitors which include without limitation 2-aminopurine; and sterol biosynthesis inhibitors which include without limitation squalene epoxidase and CYP2D6; and combinations thereof.

The one or more chemotherapeutic agent may be tested at any appropriate concentration. The concentration(s) used may be selected based on the known range of concentrations used clinically. Thus, different chemotherapeutic agents may be tested at different concentrations. Selection of appropriate concentrations is within the routine practice of one of ordinary skill in the art. Typically the one or more chemotherapeutic agent is tested at a range of concentrations (e.g. at one, two, three, four, five, six, seven, eight, nine, ten or more different concentrations), to allow the sensitivity of the cancer cells within the sample to be determined.

The efficacy of a chemotherapeutic agent or combination thereof may be determined and/or quantified by any appropriate means. Suitable techniques are known in the art. Non-limiting examples include cytotoxicity assay, cell viability assay, fluorescent imaging, direct imaging and/or fluorescence measurement. In some preferred embodiments, efficacy of a chemotherapeutic agent or combination thereof is determined by cytotoxicity and/or cell viability. Exemplary cell viability and cytotoxicity quantification techniques are described above and can readily be used to predict responsiveness to chemotherapeutic agents.

A subject may be predicted as responsive to a chemotherapeutic agent or combination thereof if the efficacy of said chemotherapeutic agent or combination thereof is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% efficacious against the CTCs within the one or more population of CTCs, and/or the CSCs and/or tumour marker⁺ cells within the one or more population of CSCs and/or tumour marker⁺ cells.

The efficacy of a chemotherapeutic agent or combination thereof may be quantified in absolute terms or relative to a control, as described above. Thus, the efficacy (e.g. as measured using cell viability and/or cytotoxicity) of a chemotherapeutic agent or combination thereof against the CTCs within the one or more population of CTCs (and/or the CSCs and/or tumour marker⁺ cells within the one or more population of CSCs and/or tumour marker⁺ cells) may be compared with the efficacy (e.g. as measured using cell viability and/or cytotoxicity) of a chemotherapeutic agent or combination thereof against a control. Typically the control is an equivalent population or sample in which no chemotherapeutic agent (or combination thereof) has been added, for example a population of CTCs (and/or CSCs and/or tumour marker⁺ cells) obtained from the sample (or a different sample from the same subject) to which the chemotherapeutic agent (or combination thereof) has not been administered.

A subject may be predicted as responsive to a chemotherapeutic agent or combination thereof if said chemotherapeutic agent or combination thereof reduces the cell viability of the CTCs (and/or the CSCs and/or tumour marker⁺ cells) within the one or more population of CTCs by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% reduction in CTCs viability within the one or more population of CTCs (and/or the CSCs and/or tumour marker⁺ cells within the one or more population of CSCs and/or tumour marker⁺ cells). This reduction may be relative to a control, as described above.

Alternatively or in addition, a subject may be predicted as responsive to a chemotherapeutic agent or combination thereof if said chemotherapeutic agent or combination thereof results in an increase in cell death of the CTCs (and/or the CSCs and/or tumour marker⁺ cells) within the one or more population of CTCs (and/or the CSCs and/or tumour marker⁺ cells) by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% increase in cell death of the CTCs within the one or more population of CTCs (and/or the CSCs and/or tumour marker⁺ cells within the one or more population of CSCs and/or tumour marker⁺ cells).

The CTCs used in said method are typically isolated from a blood sample, with each subject providing one or more blood sample as described herein. Accordingly, a method of the invention may further comprise a step of isolating CD45⁺ cells and/or CTCs from a sample, as described herein. CTCs used in a method of the invention may be less than about 20 μm in diameter, such as less than about 20 μm in diameter, less than about 19 μm in diameter, less than about 18 μm in diameter, less than about 17 μm in diameter, less than about 16 μm in diameter, less than about 15 μm in diameter, less than about 14 μm in diameter, less than about 13 μm in diameter, less than about 12 μm in diameter, less than about 11 μm in diameter, less than about 10 μm in diameter or less. Preferably CTCs used in a method of the invention are less than 20 μm in diameter. In some embodiments, the average diameter of CTCs used in methods of the invention is between about 10 μm to about 20 μm, such as between about 10 μm to about 15 μm or between about 12 μm to about 15 μm. In some embodiments, the average diameter of CTCs used in methods of the invention is about 12.4 μm.

In some preferred embodiments, CD45⁺ cells used in a method of the invention may be less than 20 μm in diameter and CTCs used in a method of the invention may be less than 20 μm in diameter.

Size of the CD45⁺ cells and/or CTCs may be determined using any appropriate technique, examples of which are known in the art, such as flow cytometry.

The CSCs and/or tumour marker⁺ cells used in said method are typically isolated from a tissue sample, with each subject providing one or more tissue sample as described herein. Accordingly, a method of the invention may further comprise a step of isolating CSCs and/or tumour marker⁺ cells from a sample, as described herein.

Where a subject provides a tissue sample (e.g. a sample comprising or consisting of cancerous tissue, such as a tissue biopsy) in addition to a blood sample, a method of predicting responsiveness of a subject having cancer to one or more chemotherapeutic agent comprising the steps of contacting at least one population of CTCs from said subject with at least one chemotherapeutic agent and determining the efficacy of the at least one chemotherapeutic agent against the CTCs according to the invention may further comprise the steps of: contacting at least one population of CSCs and/or tumour marker⁺ cells; with at least one chemotherapeutic agent or combination thereof; and determining the efficacy of the at least one chemotherapeutic agent or combination thereof against the CSCs and/or tumour marker⁺ cells.

A population CSCs and/or tumour marker⁺ cells typically comprises, consists essentially of, or consist of CSCs and/or tumour marker⁺ cells.

A plurality of populations of CSCs and/or tumour marker⁺ cells, e.g. at least two, at least three, at least four, at least five, at least six, at least seven, at least eight, at least nine, at least 10, or more populations may be contacted with a chemotherapeutic agent or combination thereof in a method of the invention.

A plurality of populations of CSCs and/or tumour marker⁺ cells may be from a single subject or from multiple (e.g. 2, 3, 4, 5, 6, 7, 8, 9, 10, 15, 20, 25 or more) subjects. Typically any individual population of CSCs and/or tumour marker⁺ cells within the plurality is from a single subject, even when the plurality comprises populations from different subjects.

Each population of CSCs and/or tumour marker⁺ cells within a plurality of populations of CSCs and/or tumour marker⁺ cells may be contacted with a separate (i.e. different) chemotherapeutic agent or combination thereof. In other words, each chemotherapeutic agent or combination thereof may be used to contact a separate (i.e. different) population of CSCs and/or tumour marker⁺ cells within a plurality of CSCs and/or tumour marker⁺ cells populations. Alternatively, each chemotherapeutic agent or combination thereof may be used to contact multiple (e.g. 2, 3, 4 or more) populations of CSCs and/or tumour marker⁺ cells within a plurality of CSCs and/or tumour marker⁺ cells, e.g. in order to provide experimental replicates.

Wherein a plurality of populations of CSCs and/or tumour marker⁺ cells comprises populations of CSCs and/or tumour marker⁺ cells from multiple subjects, each chemotherapeutic agent or combination thereof may be used to contact at least one population of CSCs and/or tumour marker⁺ cells from each subject. In other words, at least one population of CSCs and/or tumour marker⁺ cells from each subject may be contacted with each chemotherapeutic agent or combination thereof.

The methods of the invention may be used to predict the responsiveness of a subject to any chemotherapeutic agent as defined herein using at least one population of CSCs and/or tumour marker⁺ cells. The disclosure herein in relation chemotherapeutic agents and combinations thereof which can be tested according to the present invention in the context of CTCs applies equally and without reservation to testing at least one population of CSCs and/or tumour marker⁺ cells. In other words, the chemotherapeutic agents and combinations thereof as described herein can also be tested on at least one population of CSCs and/or tumour marker⁺ cells. In some embodiments, the at least one chemotherapeutic agent and combinations thereof used to contact the at least one population of CSCs and/or tumour marker⁺ cells is the same as the at least one chemotherapeutic agent or combination thereof used to contact the at least one population of CTCs.

The efficacy of an chemotherapeutic agent or combination thereof against a population of CSCs and/or tumour marker⁺ cells may be determined and/or quantified by any appropriate means, as described above.

A subject may be predicted as responsive to a chemotherapeutic agent or combination thereof if the efficacy of said chemotherapeutic agent or combination thereof is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% efficacious against the CSCs and/or tumour marker⁺ cells within the one or more population of CSCs and/or tumour marker⁺ cells.

The efficacy of a chemotherapeutic agent or combination thereof may be quantified in absolute terms or relative to a control, as described above. Thus, the efficacy (e.g. as measured using cell viability and/or cytotoxicity) of a chemotherapeutic agent or combination thereof against the CSCs and/or tumour marker⁺ cells within the one or more population of CSCs and/or tumour marker⁺ cells may be compared with the efficacy (e.g. as measured using cell viability and/or cytotoxicity) of an chemotherapeutic agent or combination thereof against a control. Typically the control is an equivalent population or sample in which no chemotherapeutic agent (or combination thereof) has been added, for example a population of CSCs and/or tumour marker⁺ cells obtained from the sample (or a different sample from the same subject) to which the chemotherapeutic agent (or combination thereof) has not been administered.

A subject may be predicted as responsive to a chemotherapeutic agent or combination thereof if said chemotherapeutic agent or combination thereof reduces the cell viability of the CSCs and/or tumour marker⁺ cells within the one or more population of CSCs and/or tumour marker⁺ cells by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% reduction in CSCs and/or tumour marker⁺ cell viability within the one or more population of CSCs and/or tumour marker⁺ cells. This reduction may be relative to a control, as described above.

Alternatively or in addition, a subject may be predicted as responsive to a chemotherapeutic agent or combination thereof if said chemotherapeutic agent or combination thereof results in an increase in cell death of the CSCs and/or tumour marker⁺ cells within the one or more population of CSCs and/or tumour marker⁺ cells by at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more, up to 100% increase in cell death of the CSCs and/or tumour marker⁺ cells within the one or more population of CSCs and/or tumour marker⁺ cells.

The CSCs and/or tumour marker⁺ cells used in said method are typically isolated from a tissue sample (e.g. a sample comprising or consisting of cancerous tissue, such as a tissue biopsy), with each subject providing one or more tissue sample as described herein. Accordingly, a method of the invention may further comprise a step of isolating CSCs and/or tumour marker⁺ cells from a sample, as described herein.

The at least one population of CSCs and/or tumour marker⁺ cells may further comprise CTCs. Inclusion of CTCs in the at least one population of CSCs and/or tumour marker⁺ cells may help compensate for tissue samples which contain low amounts of CSCs and/or tumour marker⁺ cells (e.g. is a biopsy is obtained from an area of non-cancerous tissue or an area of an affected tissue or organ which comprises lower amounts of CSCs and/or tumour marker⁺ cells compared with the tissue or organ as a whole). Inclusion of CTCs in the at least population of CSCs and/or tumour marker⁺ cells allows for the effect of a chemotherapeutic (or immunotherapeutic) to be determined even for samples with low levels of CSCs and/or tumour marker⁺ cells. In such embodiments, the methods may further comprise a step of determining the efficiency of the at least one chemotherapeutic agent or combination thereof against the CTCs in addition to CSCs and/or tumour marker⁺ cells. The disclosure herein in relation to CTCs, e.g. their isolation from a blood sample of a subject, and determining the efficacy of at least one chemotherapeutic agent or combination thereof applies equally and without reservation to embodiments relating to at least one population of CTCs, CSCs and/or tumour marker⁺ cells.

The CSCs and/or tumour marker⁺ cells, preferably both the CSCs and tumour marker⁺ cells when present within populations to be contacted with the at least one chemotherapeutic agent may be present in essentially the same proportions as the CSCs and/or tumour marker⁺ cells are present in the tissue sample. By “essentially the same proportions” it is typically meant that the proportions of CSCs and/or tumour marker⁺ cells are the same as the proportions within the tissue sample, within the limits of experimental error and/or accounting for cell loss during sample processing/isolation of the cells. By way of non-limiting example, if CSCs and tumour marker⁺ cells are present at a ratio of 1:10 within the tissue sample, then populations comprising CSCs and tumour marker⁺ cells for use in methods of the invention may comprise CSCs and tumour marker⁺ cells at a ratio of about 1:10 (e.g. from about 1:8 to about 1:12). Using populations in which CSCs and/or tumour marker⁺ cells are present in essentially the same proportions as the CSCs and/or tumour marker⁺ cells are present in the tissue sample advantageously allows for the effects of the at least one chemotherapeutic agent on the CSCs and/or tumour marker⁺ cells to be investigated in an environment that replicates in vitro the environment present within the tissue sample, and hence the subject's tumour or cancer. Populations comprising essentially the same proportions of CSCs and/or tumour marker⁺ cells may be obtained from a tissue sample by isolating the CSCs and/or tumour marker⁺ cells, and dividing the isolated CSCs and/or tumour marker⁺ cells equally into populations. The number of CSCs and/or tumour marker⁺ cells may be counted prior to dividing into populations, if a certain number of CSCs and/or tumour marker⁺ cells is required per population. Typically by dividing the CSCs and/or tumour marker⁺ cells obtained from a tissue sample into equal populations, and using these populations for each test condition and control, the proportion of CSCs and/or tumour marker⁺ cells used for each test condition and control will be essentially the same as each other and essentially the same as the proportion of CSCs and/or tumour⁺ cells within the tissue sample, and hence the subject's tumour or cancer. By way of quality control, the number of different cell types (e.g. CSCs and/or tumour marker⁺ cells) may be counted to ensure that the sample can be processed according to the invention. Alternatively or in addition, the number of populations may depend on the number of chemotherapeutic agents to be tested.

All steps comprising contacting a cell population with at least one chemotherapeutic agent may be carried out simultaneously in a method of the invention. Simultaneous contacting of populations with at least one chemotherapeutic agent may be desirable, for example, when all populations are being contacted with the same at least one chemotherapeutic, or when different chemotherapeutic agents are used but each requires the same incubation time. Alternatively, the steps comprising contacting a cell population with at least one chemotherapeutic agent may be carried out sequentially. Sequential contracting of populations with at least one chemotherapeutic may be desirable, for example, when different chemotherapeutic agents are being used, and one or more requires a different incubation time. In some embodiments, two or more populations may be contacted with at least one chemotherapeutic agent simultaneously, with one or more additional population being contacted with at least one chemotherapeutic agent (typically a different chemotherapeutic agent) sequentially.

A method of the invention may reduce the time taken to predict responsiveness of a subject to one or more chemotherapeutic agent. By way of non-limiting example, a method of the invention may reduce the time taken to predict responsiveness to one or more chemotherapeutic agent by at least 1 day, at least 2 days, at least 3 days, at least 4 days, at least 5 days, at least 6 days, at least 7 days, at least 8 days, at least 9 days, at least 10 days, at least 2 weeks, at least 3 weeks, at least 4 weeks or more. Typically such a reduction in time is compared with suitable control, such as a conventional screening method, including those used in current standards of care (e.g. within the NHS).

A method of the invention may reduce the time taken to predict responsiveness of a subject to one or more chemotherapeutic agent. By way of non-limiting example, the time taken to predict responsiveness to one or more chemotherapeutic agent using a method of the invention may be 20 days or less, 15 days or less, 14 days or less, 13 days or less, 12 days or less, 11 days or less, or 10 days or less. Preferably the time taken to predict responsiveness to one or more chemotherapeutic agent using a method of the invention is 15 days or less more preferably 10 days or less. The time taken to predict responsiveness to one or more chemotherapeutic agent using a method of the invention may be between about 4 to about 7 days, such as about 4 or 5 days.

A method of the invention may increase the success rate of correctly predicting responsiveness of a subject to one or more chemotherapeutic agent. By way of non-limiting example, a method of the invention may increase the success rate 10%, at least 20%, at least 30%, at least 40% at least 50% at least 60%, at least 70%, at least 80%, or more. Typically such an increase in the success rate is compared with suitable control, such as a conventional screening method, including those used in current standards of care (e.g. within the NHS).

A method of the invention may have a success rate of correctly predicting responsiveness of a subject to one or more chemotherapeutic agent of at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more. Preferably the success rate of a method of the invention is at least 75%, more preferably at least 80%, even more preferably at least 90%.

A method of the invention typically allows for predicting responsiveness of a subject having cancer to one or more chemotherapeutic agent, by directly assessing the activity of the one or more chemotherapeutic agent against CTCs and/or CSCs from said subject. Said method may comprise the steps of investigating the activity of the one or more chemotherapeutic agent against CTCs and/or CSCs from said subject contacted with the one or more chemotherapeutic agent and determining the efficacy of the one or more chemotherapeutic agent against the CTCs and/or CSCs. Thus, the methods of the invention focus on the activity the one or more chemotherapeutic agent against CTCs and/or CSCs, rather than physical properties (e.g. size or diameter) or other characteristics of these cell types.

Screening for Responsiveness to both Immunotherapeutic and Chemotherapeutic Agents

The invention advantageously provides methods which allow for a subject's responsiveness to both immunotherapeutic agents and chemotherapeutic agents to be predicted in parallel. In other words, the methods described above for predicting responsiveness to immunotherapeutic agents and chemotherapeutic agents can be carried out at the same time, and even as part of the same assay protocol/set up.

Accordingly, the invention provides a method of predicting responsiveness of a subject having cancer to one or more immunotherapeutic agent and one or more chemotherapeutic agent, said method comprising the steps of: (a) contacting at least one population of CD45⁺ cells and CTCs from said subject with at least one immunotherapeutic agent; (b) contacting at least one population of CTCs from said subject with at least one chemotherapeutic agent; and (c) determining the efficacy of the at least one immunotherapeutic agent and at least one therapeutic agent against the CTCs. Said method is typically used using a blood sample provided by the subject.

The invention provides a method of predicting responsiveness of a subject having cancer to one or more immunotherapeutic agent and one or more chemotherapeutic agent, said method comprising the steps of: (a) contacting at least one population of (i) CD45⁺ cells and (ii) CSCs and/or tumour marker⁺ cells from said subject with at least one immunotherapeutic agent; (b) contacting at least one population of CSCs and/or tumour marker⁺ cells from said subject with at least one chemotherapeutic agent; and (c) determining the efficacy of the at least one immunotherapeutic agent and at least one therapeutic agent against the CSCs and/or tumour marker⁺ cells. Said method is typically used using a tissue sample provided by the subject.

Any and all of the disclosure above in relation to methods for predicting a subject's responsiveness to both immunotherapeutic agents and chemotherapeutic agents applies equally and without reservation to such combined methods. Any and all features and embodiments relating to methods for predicting a subject's responsiveness to immunotherapeutic agents may be combined with any and all features and embodiments relating to methods for predicting a subject's responsiveness to chemotherapeutic agents.

Thus, in a method of predicting responsiveness of a subject having cancer to one or more immunotherapeutic agent and one or more chemotherapeutic agent according to the invention: (i) each of the at least one immunotherapeutic agents may contacted with a separate population of CD45⁺ cells and CTCs; and/or (ii) each of the at least one chemotherapeutic agents may contacted with a separate population of CTCs.

Said method may further comprises the steps of: (d) contacting at least one population of CD45⁺ cells, CTCs, cancer stem cells (CSCs) and tumour marker⁺ cells with at least one immunotherapeutic agent; and (e) determining the efficacy of the at least one immunotherapeutic agent against the CTCs, CSCs and/or tumour marker⁺ cells. Optionally the at least one immunotherapeutic agent is the same at least one immunotherapeutic agent as used in step (a).

Alternatively or in addition to further comprising steps (d) and (e), said method may further comprise the steps of: (f) contacting at least one population of CSCs and/or tumour marker⁺ cells with at least one chemotherapeutic agent; and (g) determining the efficacy of the at least one chemotherapeutic agent against the CSCs and/or tumour marker⁺ cells. The at least one population of CSCs and/or tumour marker⁺ cells may further comprise CTCs, and the method further comprises determining the efficacy of the at least one chemotherapeutic agent against the CTCs. The at least one chemotherapeutic agent may be the same at least one chemotherapeutic agent as used in step (b).

In a method of predicting responsiveness of a subject having cancer to one or more immunotherapeutic agent and one or more chemotherapeutic agent according to the invention: (i) each of the at least one immunotherapeutic agents may contacted with a separate population of CD45⁺ cells and CSCs and/or tumour marker⁺ cells; and/or (ii) each of the at least one chemotherapeutic agents may contacted with a separate population of CSCs and/or tumour marker⁺ cells.

All steps comprising contacting a cell population with at least one immunotherapeutic agent and/or at least one chemotherapeutic agent may be carried out simultaneously in a method of the invention. Simultaneous contacting of populations with at least one immunotherapeutic agent and/or at least one chemotherapeutic agent may be desirable, for example, when all populations are being contacted with the same at least one immunotherapeutic agent and/or at least one chemotherapeutic, or when different immunotherapeutic agents and/or chemotherapeutic agents are used but each requires the same incubation time. Alternatively, the steps comprising contacting a cell population with at least one immunotherapeutic agent and/or at least one chemotherapeutic agent may be carried out sequentially. Sequential contracting of populations with at least one immunotherapeutic agent and/or at least one chemotherapeutic may be desirable, for example, when different immunotherapeutic agents and/or chemotherapeutic agents are being used, and one or more requires a different incubation time. In some embodiments, two or more populations may be contacted with at least one immunotherapeutic agent and/or at least one chemotherapeutic agent simultaneously, with one or more additional population being contacted with at least one immunotherapeutic agent and/or at least one chemotherapeutic agent (typically a different immunotherapeutic agent and/or chemotherapeutic agent) sequentially.

A method of the invention may reduce the time taken to predict responsiveness of a subject to one or more chemotherapeutic agent. By way of non-limiting example, a method of the invention may reduce the time taken to predict responsiveness to at least one immunotherapeutic agent and/or at least one chemotherapeutic agent by at least 1 day, at least 2 days, at least 3 days, at least 4 days, at least 5 days, at least 6 days, at least 7 days, at least 8 days, at least 9 days, at least 10 days, at least 2 weeks, at least 3 weeks, at least 4 weeks or more. Typically such a reduction in time is compared with suitable control, such as a conventional screening method, including those used in current standards of care (e.g. within the NHS).

A method of the invention may reduce the time taken to predict responsiveness of a subject to at least one immunotherapeutic agent and/or at least one chemotherapeutic agent. By way of non-limiting example, the time taken to predict responsiveness to at least one immunotherapeutic agent and/or at least one chemotherapeutic agent using a method of the invention may be 20 days or less, 15 days or less, 14 days or less, 13 days or less, 12 days or less, 11 days or less, or 10 days or less. Preferably the time taken to predict responsiveness to at least one immunotherapeutic agent and/or at least one chemotherapeutic agent using a method of the invention is 15 days or less more preferably 10 days or less. The time taken to predict responsiveness to one or more immunotherapeutic agent and/or one or more chemotherapeutic agent using a method of the invention may be between about 4 to about 7 days, such as about 4 or 5 days.

A method of the invention may increase the success rate of correctly predicting responsiveness of a subject to at least one immunotherapeutic agent and/or at least one chemotherapeutic agent. By way of non-limiting example, a method of the invention may increase the success rate 10%, at least 20%, at least 30%, at least 40% at least 50% at least 60%, at least 70%, at least 80%, or more. Typically such an increase in the success rate is compared with suitable control, such as a conventional screening method, including those used in current standards of care (e.g. within the NHS).

A method of the invention may have a success rate of correctly predicting responsiveness of a subject to at least one immunotherapeutic agent and/or at least one chemotherapeutic agent of at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99% or more. Preferably the success rate of a method of the invention is at least 75%, more preferably at least 80%, even more preferably at least 90%.

A method of the invention typically allows for predicting responsiveness of a subject having cancer to one or more immunotherapeutic agent one and/or more chemotherapeutic agent, by directly assessing (i) the activity of CD45⁺ cells from said subject against CTCs and/or CSCs from said subject in the presence of one or more immunotherapeutic agent and/or (ii) the activity of the one or more chemotherapeutic agent against CTCs and/or CSCs from said subject. Said method may comprise the steps of (i) investigating the activity of at least one population of CD45⁺ cells against CTCs and/or CSCs from said subject contacted with at least one immunotherapeutic agent and determining the efficacy of the at least one immunotherapeutic agent against the CTCs and/or (ii) determining the efficacy of the one or more chemotherapeutic agent against the CTCs and/or CSCs. Thus, the methods of the invention focus on the activity of (i) the CD45⁺ cells against CTCs and/or CSCs and/or (ii) the one or more chemotherapeutic agent against CTCs and/or CSCs, rather than physical properties (e.g. size or diameter) or other characteristics of these cell types.

### Tumour Marker Negative (Tumour Marker) Cells

Any of the at least one populations of cells used in a method of the invention may further comprise tumour marker negative (tumour marker-cells). Thus, the at least one population of: (i) CD45⁺ cells and CTCs; (ii) CTCs; (iii) CD45⁺ cells, CTCs, CSCs and/or tumour marker⁺ cells; and/or (iv) CSCs and/or tumour marker⁺ cells and optionally CTCs; may further comprise tumour marker-cells.

The tumour marker cells may be defined or identified in terms of their marker expression, as described herein. Thus, the tumour marker cells may be positive or high for the expression of one or more of CD31, CD45 and/or Gly-A, or any combination thereof, as described herein. Alternatively or in addition, a tumour marker cell can be bound or detected by an anti-fibroblast antibody, again as described herein. The tumour marker cells used in said method are typically isolated from a tissue sample (e.g. a sample comprising or consisting of cancerous tissue, such as a tissue biopsy), with each subject providing one or more tissue sample as described herein. The tumour marker cells are typically isolated from the same tissue sample used to isolate the CSCs and/or tumour marker⁺ cells. Accordingly, a method of the invention may further comprise a step of isolating tumour marker cells (and optionally CSCs and/or tumour marker⁺ cells) from a sample. Any appropriate technique may be used to isolate the tumour marker cells from the sample. Such techniques are well-known in the art. Non-limiting examples of suitable techniques include magnetic activated cell sorting (MACS), fluorescence activated cell sorting (FACS), flow cytometry, and buoyancy activated cell sorting (BACS). In some preferred embodiments, MACS is used to isolate tumour marker cells from a sample. By way of non-limiting example, MACS may be used with beads with at least one binding agent for one or more of CD31, CD45 and/or Gly-A (e.g. anti-CD31, anti-CD45 and/or anti-Gly-A antibodies) and/or an anti-fibroblast antibody as described herein may be used to isolate tumour marker cells via MACS. Having isolated tumour marker cells (e.g. using MACS), the remaining cells in a tissue sample may be defined as tumour marker⁺ cells.

The tumour marker cells when present within populations to be contacted with the at least one immunotherapeutic and/or chemotherapeutic agent may be present in essentially the same proportion as the tumour marker cells are present in the tissue sample, particularly compared to the proportion of tumour marker cells relative to the CSCs and/or tumour marker⁺ cells within the tissue sample. By “essentially the same proportions” it is typically meant that the proportions of tumour marker cells (and optionally CSCs and/or tumour marker⁺ cells) are the same as the proportions within the tissue sample, within the limits of experimental error and/or accounting for cell loss during sample processing/isolation of the cells. By way of non-limiting example, if tumour marker cells and CSCs cells are present at a ratio of 5:1 within the tissue sample, then populations comprising tumour marker-cells and CSCs for use in methods of the invention may comprise tumour marker-cells and CSCs at a ratio of about 5:1 (e.g. from about 4:1 to about 6:1). By way of a further non-limiting example, if tumour marker cells, CSCs cells and tumour marker⁺ cells are present at a ratio of 5:1:10 within the tissue sample, then populations comprising tumour marker-cells, CSCs and tumour marker⁺ cells for use in methods of the invention may comprise tumour marker-cells, CSCs and tumour marker⁺ cells at a ratio of about 5:1:10 (e.g. from about 4:1:8 to about 6:1:12). Using populations in which tumour marker-cells (and optionally CSCs and/or tumour marker⁺ cells) are present in essentially the same proportions as the tumour marker cells (and optionally CSCs and/or tumour marker⁺ cells) are present in the tissue sample advantageously allows for the effects of the at least one chemotherapeutic and/or immunotherapeutic agent on the CSCs and/or tumour marker⁺ cells to be investigated in an environment that replicates in vitro the environment present within the tissue sample, and hence the subject's tumour or cancer. Populations comprising essentially the same proportions of tumour marker cells (and optionally CSCs and/or tumour marker⁺ cells) may be obtained from a tissue sample by isolating tumour marker cells (and optionally CSCs and/or tumour marker⁺ cells), and dividing the isolated tumour marker cells (and optionally CSCs and/or tumour marker⁺ cells) equally into populations. The number of tumour marker-cells (and optionally CSCs and/or tumour marker⁺ cells) may be counted prior to dividing into populations, if a certain number of tumour marker cells (and optionally CSCs and/or tumour marker⁺ cells) is required per population. Typically by dividing the CSCs and/or tumour marker⁺ cells obtained from a tissue sample into equal populations, and using these populations for each test condition and control, the proportion of CSCs and/or tumour marker⁺ cells used for each test condition and control will be essentially the same as each other and essentially the same as the proportion of CSCs and/or tumour⁺ cells within the tissue sample, and hence the subject's tumour or cancer. By way of quality control, the number of different cell types (e.g. CSCs and/or tumour marker⁺ cells) may be counted to ensure that the sample can be processed according to the invention. [Alternatively or in addition, the number of populations may depend on the number of immunotherapeutic agents to be tested.

### Scaffolds

Any of the at least one populations of cells used in a method of the invention may be plated onto a biocompatible scaffold. Typically plating occurs prior to contacting a population with at least one immunotherapeutic agent and/or at least one chemotherapeutic agent. Thus, the at least one population of: (i) CD45⁺ cells and CTCs; (ii) CTCs; (iii) CD45⁺ cells, CTCs, CSCs and/or tumour marker⁺ cells; and/or (iv) CSCs and/or tumour marker⁺ cells and optionally CTCs; any of (i)-(iv) optionally further comprising tumour marker cells may be plated onto a biocompatible scaffold prior to contacting with at least one immunotherapeutic agent and/or at least one chemotherapeutic agent.

By plating, it is meant that the cells of a population are cultured on or in the presence of the scaffold, or otherwise contacted with the scaffold. Preferably, the cells of a population are seeded onto a scaffold prior to contacting with the at least one immunotherapeutic agent and/or at least one chemotherapeutic agent.

The scaffold may be made of a defined biocompatible material. For example, the scaffold may comprise or consist of extracellular matrix (ECM) protein to improve the adhesiveness of the cells. The scaffold can comprise or consist of any material intended to attach cells, such as collagen, gelatin, poly-L-lysine, poly-D-lysine, laminin, and fibronectin, fragments or mixtures thereof.

Typically, the biocompatible scaffold comprises or consists of a layer of biocompatible electrospun polymer fibers. Non-limiting examples of suitable electrospun polymer fibers may comprise or consist of a polymer selected from the group consisting of polycaprolactone, polyethylene terephthalate, silicone, polyurethane, polycarbonate, polyether ketone,, polylactic acid, polyglycolic acid, collagen, gelatin, fibronectin, hyaluronic acid, and combinations thereof, preferably polycaprolactone. Suitable examples of biocompatible scaffolds comprising electrospun polymer fibres are described in US20190153398, U.S. Pat. Nos. 10,653,635, 9,737,632, 10,562,225, US20210138104, EP2971318, U.S. Pat. Nos. 10,898,608, 10,166,315, US20190269829, EP3288602 and EP3370788, each of which is herein incorporated by reference in its entirety.

### Drug Response Score

Preferably a subject's responsiveness to any chemotherapeutic agent and/or immunotherapeutic agent is determined using a drug response score (DRS), also referred to as a drug response metric (DRM).

Examples of suitable DRS models and means for calculating DRSs are known in the art and it is within the routine practice of one of ordinary skill in the art to select a suitable DRS for determining and/or quantifying a subject's response to a chemotherapeutic agent and/or immunotherapeutic agent.

Preferably, a normalised drug response (NDR) score or metric may be used. By way of non-limiting example, one such NDR calculation is:

$$NDR = max ( - 1 , \frac{1 - {2}^{( l {og}_{2} ( {foldChange}_{drug} ) / l {og}_{2} ( {foldChange}_{posCtrl} ) )}}{1 - {2}^{( l {og}_{2} ( {foldChange}_{negCtrl} ) / l {og}_{2} ( {foldChange}_{posCtrl} ) )}} )$$

The fold change between the readouts at the start and end-points of the measurement may be calculated as:

$${foldChange}_{condn} = \frac{{End\_Readout}_{condn}}{{Start\_Readout}_{condn}}$$

Whilst the above calculation specifies a log ₂ scaling, any other logarithmic base constant (e.g. log ₁₀) may be used instead.

The start readout for a given immunotherapeutic or chemotherapeutic agent at a given concentration is typically the to readout (e.g. immediately preceding or following addition of immunotherapeutic or chemotherapeutic agent) for said immunotherapeutic or chemotherapeutic agent at a specified concentration. The end readout for a given immunotherapeutic or chemotherapeutic agent at a given concentration is typically the final timepoint reading (e.g. t₉₆ₕ or t₄₈ₕ) for said immunotherapeutic or chemotherapeutic agent at a specified concentration.

The positive control start readout for a given immunotherapeutic or chemotherapeutic agent at a given concentration may be the to readout for the corresponding immunotherapeutic or chemotherapeutic agent at said given concentration. By way of non-limiting example, the positive control start readout for gemcitabine at a concentration of 10 μg/ml may be the to readout for gemcitabine at a concentration of 10 μg/ml. The positive control end readout for the corresponding given immunotherapeutic or chemotherapeutic agent at a given concentration may be the final timepoint reading (e.g. t₉₆ₕ or t₄₈ₕ) for a blank well (i.e. corresponding to a well with zero cells).

The negative control start and end readouts for a given immunotherapeutic or chemotherapeutic agent at a given concentration may be the to and final timepoint reading (e.g. t₉₆ₕ or t₄₈ₕ) respectively for blank cell well (i.e. corresponding to a well containing the same cell populations as the wells contacted with a given immunotherapeutic or chemotherapeutic agent at a given concentration to be tested, but without contacting the cells of the negative control with the given immunotherapeutic or chemotherapeutic agent at the given concentration—the negative controls are referred to as “blank cell” or “blank immuno-cell” wells in the Examples herein).

The specific fold change values may be selected to improve results, e.g. average (median or mean) values may be used to make the NDR calculation less sensitive to outlier values.

Based on these NDR values, the efficacy of a chemotherapeutic and/or immunotherapeutic agent may be classified as follows:

| Drug Response Score | Interpretation |
|---|---|
| >1.0 | Resistant (−) |
| 0.0 to 1.0 | Effective (+) |
| −1.0 to 0.0 | Very Effective (++) |
| <−1.0 | Complete Clearance (+++) |

This NDR model and calculation are described in more detail in Gupta et al. (Communications Biology doi: 10.1038/s42003-020-0765-z), which is herein incorporated by reference in its entirety.

Optimised/modified forms of this NDR calculation may also be used according to the present invention.

### Further Steps

A method of the invention may further comprise one or more additional step. Such steps may include isolation of cells from a sample (as described herein), enriching cells or populations thereof, expanding cells or populations thereof, and/or culturing cells or populations thereof.

Any additional steps of enriching, expanding and/or culturing cells may be carried out in any suitable culture system, or using any suitable culture medium. Such culture systems and/or media may be xeno-free (i.e. free of animal components or components from non-human The medium according to the present invention may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolglycerol, or equivalents thereto. Alternatively, any commercially available materials can be used for more convenience. The commercially available materials include knockout Serum Replacement (KS®), Chemically-defined Lipid concentrated (Gibco), and Glutamax (Gibco).

Any suitable container, flask, or appropriate tube such as a 96 well plate, 24 well plate, 12.5 cm² T flask or gas-permeable bag can be used in the method of this invention. Such culture-containers are commercially available from Falcon, Corning or Costar. A culture vessel used for the methods of the invention and/or for expanding cells can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing cells therein.

Any of the cell types described herein may be expanded prior to use in a method of predicting responsiveness of a subject to an immunotherapeutic agent and/or chemotherapeutic agent. Such expansion may be carried out in an “expansion container”, which is intended to include any chamber or container for expanding cells whether or not free standing or incorporated into an expansion apparatus such as a bioreactor.

Various media can be used for the enrichment, expansion and/or culture of any of the cell types described herein. Illustrative media include Dulbecco's MEM (DMEM), IMDM and RPMI-1640 that can be supplemented with a variety of different nutrients, growth factors, cytokines, etc. The medium can also contain fatty acids or lipids, amino acids (such as non-essential amino acids), vitamin(s), growth factors, cytokines, antioxidant substances, 2-mercaptoethanol, pyruvic acid, buffering agents, and inorganic salts. The media can be serum free or supplemented with suitable amounts of serum such as fetal calf serum (FCS, also referred to as fetal bovine serum, FBS) or autologous serum. One suitable medium is one containing DMEM, effective amounts of at FBS, L-glutamine, insulin and epidermal growth factor (EGF).

Other culturing conditions can be appropriately defined. For example, the culturing temperature can be about 30 to 40° C., for example, at least or about 31, 32, 33, 34, 35, 36, 37, 38, 39° C. but particularly not limited to them. Preferably the culture temperature is around 37° C. The CO2 concentration can be about 1 to 10%, for example, about 2 to 5%, or any range derivable therein, preferably around 5%. The oxygen tension can be at least, up to, or about 1, 2, 3, 4, 5, 6, 7, 8, 9, 10, 20%, or any range derivable therein.

The cells (any of the cell types described herein or populations comprising or consisting thereof) may be grown for around 1, 2, 3, 4, 5, 6, 7, 8, 9, 10, 11, 12, 13, 14, 15, 16, 17, 18, 19, 20, 21, 22, 23, 24, 25 days or any range derivable therein prior to contacting with at least one immunotherapeutic agent and/or at least one chemotherapeutic agent. Alternatively, the cell populations may be plated onto the scaffold for only a short period (e.g. between about 5 minutes to about 12 hours, such as between about 15 minutes to about 90 minutes, particularly between about 5 minutes to about 20 minutes, or between about 20 minutes to about 1 hour) prior to contacting with the at least one immunotherapeutic agent and/or at least one chemotherapeutic agent. This shorter period typically allows for attachment of the cells to the scaffold, but does not allow for (or allows minimal) cell expansion.

Following contacting of the cell populations with the at least one immunotherapeutic agent and/or at least one chemotherapeutic agent, the cell populations may be cultured (incubated) to allow for the effects of said agents to be observed. Any appropriate culture/incubation time may be used, provided that it sufficient to observe the effect of the at least one immunotherapeutic agent and/or at least one chemotherapeutic agent. Typically, the incubation period (including or excluding the step of plating the cell populations on a scaffold prior to contacting with the at least one immunotherapeutic agent and/or at least one chemotherapeutic agent) may be between about 12 hours to about 2 weeks, such as between about 1 day to about 7 days, or between about 2 days to about 7 days. The incubation period (including or excluding the step of plating the cell populations on a scaffold prior to contacting with the at least one immunotherapeutic agent and/or at least one chemotherapeutic agent), may be about 1 day, about 2 days, about 3 days about 4 days about 5 days, about 6 days or about 7 days, preferably about 4 or 5 days.

The cell populations may be contacted with the at least one immunotherapeutic agent and/or at least one chemotherapeutic agent multiple times, e.g. two times, three times, four times or more. Preferably, the cell populations are contacted with the at least one immunotherapeutic agent and/or at least one chemotherapeutic agent for between about 1 to 3 days (e.g. 2 days), following which an initial determination and/or quantification of the efficacy of the at least one immunotherapeutic agent and/or at least one chemotherapeutic agent may be made (using any appropriate means as described here). The cell populations may then be contacted for a second time with at least one immunotherapeutic agent and/or at least one chemotherapeutic agent (typically the same at least one immunotherapeutic agent and/or at least one chemotherapeutic agent as used for the first contacting) for a further period of between about 1 to 3 days (e.g. 2 days), following which a second determination and/or quantification of the efficacy of the at least one immunotherapeutic agent and/or at least one chemotherapeutic agent may be made (using any appropriate means as described here). This process can be repeated as many times as desired to assess the effects of multiple contacting steps.

### Methods of Treatment

The invention further provides a method of treating cancer in a subject in need thereof, said method comprising: (a) carrying out a method as defined herein to predict the responsiveness of the subject to one or more immunotherapeutic agent and/or one or more chemotherapeutic agent; and (b) administering an effective amount of the one or more immunotherapeutic agent and/or one or more chemotherapeutic agent to the subject.

Methods of treatment using one or more immunotherapeutic agent and/or one or more chemotherapeutic agent as described herein may be used in further combination with other known therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

It will be appreciated that appropriate dosage of the to one or more immunotherapeutic agent and/or one or more chemotherapeutic agent, will depend on the specific agent, and can also vary from patient to patient.

Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

### Other Methods

The invention provides a method of selecting a subject with cancer for treatment, said method comprising: (a) carrying out a method as herein to predict the responsiveness of the subject to one or more immunotherapeutic agent and/or one or more chemotherapeutic agent; and (b) selecting the subject for treatment on the basis of their responsiveness to one or more immunotherapeutic agent and/or one or more chemotherapeutic agent.

The invention also provides a method of selecting an immunotherapeutic agent and/or chemotherapeutic agent for treating a subject with cancer, said method comprising: (a) carrying out a method as defined herein to predict the responsiveness of the subject to one or more immunotherapeutic agent and/or one or more chemotherapeutic agent; and (b) selecting an immunotherapeutic agent and/or chemotherapeutic agent for treatment on the basis of the subject's responsiveness to the one or more immunotherapeutic agent and/or one or more chemotherapeutic agent.

### Cancer

The methods described herein can be used with any cancer cell or in a subject having any type of cancer, for example those described by the National Cancer Institute. The cancer can be a carcinoma, a sarcoma, or any solid cancer. Typically the methods described herein are used to screen agents for treating solid cancers, particularly when the method is carried out using cells isolated from a tissue sample from a subject. Exemplary solid cancers described by the National Cancer Institute include but are not limited to: lung cancer, breast cancer, colon cancer, prostate cancer, melanoma (skin cancer), kidney cancer (renal cell carcinoma), head and neck cancer (squamous cell carcinoma), pancreatic cancer, brain or CNS cancer, bladder cancer, oesophageal cancer, cancer of unknown primary, ovarian cancer, stomach cancer, liver cancer, thyroid cancer and uterine cancer.

Lung cancer such as non-small cell lung cancer; and small cell lung cancer are particularly preferred.

Respiratory cancers such as adult malignant mesothelioma; childhood malignant mesothelioma; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;
Digestive/gastro intestinal cancers such as anal cancer; bile duct cancer; extrahepatic bile duct cancer; appendix cancer; carcinoid tumour, gastrointestinal cancer; colon cancer; colorectal cancer including childhood colorectal cancer; oesophageal cancer including childhood oesophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;
Endocrine cancers such as islet cell carcinoma (endocrine pancreas); adrenocortical carcinoma including childhood adrenocortical carcinoma; gastrointestinal carcinoid tumour; parathyroid cancer; pheochromocytoma; pituitary tumour; thyroid cancer including childhood thyroid cancer; childhood multiple endocrine neoplasia syndrome; and childhood carcinoid tumour;
Eye cancers such as intraocular melanoma; and retinoblastoma;
Musculoskeletal cancers such as Ewing's family of tumours; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;
Breast cancer such as breast cancer including childhood and male breast cancer and breast cancer in pregnancy;
Neurologic cancers such as childhood brainstem glioma; brain tumour; childhood cerebellar astrocytoma; childhood cerebral astrocytoma/malignant glioma; childhood ependymoma; childhood medulloblastoma; childhood pineal and supratentorial primitive neuroectodermal tumours; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumours; central nervous system atypical teratoid/rhabdoid tumour; central nervous system embryonal tumours; and childhood supratentorial primitive neuroectodermal tumours and pituitary tumour;
Genitourinary cancers such as bladder cancer including childhood bladder cancer; renal cell (kidney) cancer; ovarian cancer including childhood ovarian cancer; ovarian epithelial cancer; ovarian low malignant potential tumour; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumour and other childhood kidney tumours; endometrial cancer; and gestational trophoblastic tumour; Germ cell cancers such as childhood extracranial germ cell tumor; extragonadal germ cell tumour; ovarian germ cell tumour;
Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer; Skin cancers such as Kaposi's sarcoma; Merkel cell carcinoma; melanoma; and childhood skin cancer;
AIDS-related malignancies; and
Other childhood cancers, unusual cancers of childhood and cancers of unknown primary site.

The invention may relate to screening for responsiveness of the aforementioned cancers and metastases thereof according to the present invention. Said aforementioned cancers and metastases thereof may also be treated or prevented in accordance with the methods described herein.

In some preferred embodiments, the methods described herein may be suited for lung (e.g., small cell lung), bladder, testicular, ovarian, head and neck, cervical, mesothelioma, oesophageal, melanoma, brain tumour, neuroblastoma, colorectal, Wilms' tumour, retinoblastoma, breast, endometrial, adrenocortical, anal, biliary tract, carcinoid tumours, choriocarcinoma, gastric, liver cancer, osteosarcoma, soft-tissue sarcomas, penile, malignant thymoma, anaplastic thyroid cancer, rhabdoid tumour of the kidney, advanced medullary thyroid cancer, carcinoid, mesothelioma, bone, gliomas, squamous cell carcinoma, pancreatic or prostate cancers.

In some preferred embodiments, the methods may be used for lung cancer (e.g., small cell lung cancer or non-small cell lung cancer), bladder cancer (e.g., muscle-invasive bladder carcinoma, advanced or metastatic bladder carcinoma), testicular cancer (e.g., non-seminomatous testicular carcinoma, disseminated seminoma testis or extragonadal germ-cell tumours), ovarian cancer (e.g., ovarian epithelial cancer or ovarian germ-cell tumours), head and neck cancer (e.g., squamous cell carcinoma), breast cancer, pancreatic cancer, sarcomas, cervical cancer (e.g., invasive, metastatic or recurrent cervical cancer), Wilms' tumour, brain tumours (e.g., gliomas, medulloblastoma or germ cell tumours), neuroblastoma, retinoblastoma, mesothelioma (e.g., malignant pleural mesothelioma), oesophageal cancer (e.g., localized or advanced oesophageal cancer), melanoma, and colorectal cancer.

### Samples

Any tissue, or bodily fluid may be utilised to obtain cells for use in a method of the invention. Such tissues, and fluid may include sections of tissues such as biopsy and autopsy samples, frozen sections taken for histologic purposes, blood, plasma, serum, sputum, stool, tears, mucus, saliva, hair, and skin. Tissues may also include broncho-lavage, samples from endobronchial ultrasound (EBUS), lymph fluid, ascetic fluid, gynaecological fluid, urine, peritoneal fluid, cerebrospinal fluid, a fluid collected by vaginal rinsing, or a fluid collected by vaginal flushing.

A tissue or cell type may be provided by removing a sample of cells from a subject as part of the method. Alternatively, a method of the invention may use previously isolated cells (e.g., isolated by another person, at another time, and/or for another purpose). Archival tissues, such as those having treatment or outcome history, may also be used.

Tissue samples are typically standard clinical samples used for cancer diagnosis/prognosis. Thus, standard tissue sample types and/or volumes are typically used. By way of non-limiting example, standard biopsies may have a diameter of between about 0.5 mm to about 5 mm. A standard biopsy may have a diameter of about 5 mm or less, about 4 mm or less, about 3 mm or less, about 2 mm or less, about 1 mm or less, or about 0.5 mm or less. A biopsy of diameter of about 1 mm may be used according to the invention. Alternatively or in addition, standard biopsies may have a length of from about 0.5 cm to about 10 cm, such as from about 0.5 cm to about 7.5 cm, from about 0.5 cm to about 5 cm or from about 1 cm to about 5 cm. A biopsy of length of about 1 cm to about 5 cm may be used according to the invention. A biopsy of diameter of between about 0.5 mm to about 5 mm (e.g. between about 0.5 mm to about 2 mm) and a length of between about 0.5 cm to about 10 cm in length (e.g. between about 1 cm to about 5 cm) may be used according to the invention. A biopsy of diameter of about 1 mm and about 1 cm to about 5 cm in length may be used according to the invention.

Typically, the methods described herein use cells, particularly CD45⁺ cells and/or CTCs isolated from a blood sample of a subject. Said blood sample is typically a whole blood sample. The suitable volume of a blood sample could be from about 1 to about 5 ml, about 1 to 10 ml, about 1 to 15 ml, or more specifically, about 3, 4, 5, 6, 7, 8, 9, 10 ml or any range derivable therein, preferably 4 ml. The cells may be obtained from a cryopreserved blood sample or the cells may have been cryopreserved.

Preferably tissue and/or blood samples according to the present invention have not been frozen prior to use in a method of the invention.

CSCs, tumour marker⁺ cells and/or tumour marker cells may be isolated from a tissue sample of a subject. Said tissue sample is typically a sample comprising or consisting of cancerous tissue. Examples of tissue samples include tissue biopsies, such as those that may be taken by or on the instruction of the responsible clinician for diagnostic purposes.

The sample may be used directly as obtained from the subject or following pre-treatment to modify a character of the sample. Pre-treatment may include disaggregation of tissue; extraction, expansion, isolation, enrichment, and/or concentration of cells; inactivation of interfering components; storage; freezing; and/or the addition of reagents.

### Kits

The invention provides a kit for use in the method of the invention, said kit comprising: (i) a biocompatible scaffold as defined herein, optionally attached to an inert support; (ii) means for isolating each of CD45⁺ cells, CTCs, CSCs, tumour marker⁺ cells and/or tumour marker-cells (optionally MACS beads with a suitable binding agent, as described herein); and/or (iii) one or more agent for determining cell viability and/or cytotoxicity. Said kit may further comprise one or more additional agent or component, as described herein.

Components of a kit are generally sterile and in sealed vials or other containers. Kits may be employed in therapy, diagnostic analysis or other applications as described herein.

A kit may contain instructions for use of the components, e.g., for a treatment or method in accordance with the present invention. Ancillary materials to assist in or to enable performing such a treatment or method may be included within a kit of the invention.

Each component of the kits is generally in its own suitable container. Thus, these kits generally comprise distinct containers suitable for each component (each binding member present). Further, the kits may comprise instructions for performing the treatment or method, and/or for interpreting and analysing data resulting from the performance of the treatment or method.

### Examples

The invention will be further clarified by the following examples, which are intended to be purely exemplary of the invention and are in no way limiting.

### Methods

Blood and/or tissue samples from individual patients were processed and analysed.

### Blood Samples

CD45⁺ cells and circulating tumour cells (CTCs) were isolated from a blood sample provided by each subject.

CD45 separation was performed by adding human CD45 microbeads (50 μl/ml blood), incubating for 15 minutes at 4° C. and then processing using an AutoMACS machine. This separates the blood sample into CD45⁺ and CD45⁻cells in 2 separate falcons. The falcon tubes are then centrifuged at 1200 rpm for 5 minutes.

The supernatant was removed from the CD45⁺falcon tube, and the cells resuspended in DMEM with 2 μl Cell Tox, 2 μl MT and 2 μl MT. 20 μl of the resuspended CD45⁺ cells was then plated in each well which will contain an immunotherapy treatment.

The supernatant was removed from the CD45⁻falcon tube, and the CD45⁻cells contacted with 100 μl of FcR block and 100 μl of CD326 (EpCAM) MicroBeads, followed by incubation at are added, this is then incubated at 4° C. for 30 minutes. The CD45⁻cells were then processed using an AutoMACS machine to separate the EpCAM positive and negative cells into 2 separate falcon tubes. The EpCAM-cells were discarded. The supernatant was removed from the EpCAM⁺ cells, and the cells resuspended in DMEM with 2 μl Cell Tox, 2 μl MT and 2 μl MT. 20 μl of the resuspended pellet was then added to all sample wells excluding a blank. After all wells are added to, a cell count was conducted on the EpCAM-fraction to determine the number of circulating tumour cells (CTCs).

### Tissue Samples

Where a tissue sample was provided by each subject, cancer stem cells (CSCs, CD133⁺), were isolated from said sample. Tumour marker cells were further isolated from each tissue sample. The remaining cells were designated tumour marker⁺ cells.

First, disaggregation of the tissue samples was carried out. Disaggregation was dependent on the size of the tissue sample and the method of retrieval:
EBUS (Endobronchial Ultrasound Scan and Biopsy)—very small fragments of tissue in solution-spun at 1200 rpm for 5 minutes to collect tissue pellet
Bronchoscopy-larger fragments with larger amount of blood vessels
Broncho-lavage-similar to EBUS, spun at 1200 rpm for 5 minutes to collet tissue pellet

Once a pellet of tissue obtained, DMEM added to the tissue pellet and the material forced through a 70 uM Smart strainer. Once a cell suspension was achieved, this was spun at 1200 rpm for 5 minutes and the supernatant removed. The cells were then resuspended to a volume of 60 μl.

20 μl of FcR block and 20 μl of CD133 microbeads were added to the resuspended cells, followed by incubation for 15 minutes at 4° C. The cells were then processed using an AutoMACS machine. This separates the blood sample into CD133⁺ and CD133⁻ cells in 2 separate falcons. The falcon tubes are then centrifuged at 1200 rpm for 5 minutes.

The supernatant was removed from the CD133+falcon tube, and the cells resuspended in DMEM with 2 μl Cell Tox, 2 μl MT and 2 μl MT. 20 μl of the resuspended CD45⁺ cells was then plated in each well which will contain an immunotherapy treatment.

The supernatant was removed from the CD133⁻ falcon tube, and the CD133⁻ cells contacted with 40 μl of a tumour separation microbeads cocktail containing microbeads bound to anti-CD31, anti-CD45, anti-Glycophorin-A (Gly-A) and an anti-fibroblast antibody.

This cocktail is used to separate out tumour marker cells using MACS, by separating out CD31⁺, CD45⁺ and Gly-A⁺ cells. In addition, anti-fibroblast antibody-labelled beads are also used. The cocktail is added to the CD133⁻ falcon tube, followed by incubation for 15 minutes at 4° C. MACS is carried out using an AutoMACS machine. This cells which are not bound by the CD31, CD45, Gly-A and anti-fibroblast antibody beads are the tumour marker⁺ cells.

### Analysis

The different cell types were each divided equally into populations, depending on the number of drugs/concentrations to be tested and plated on a cell-culture plate pre-coated with a scaffold of polycaprolactone (PCL) electrospun fibres and incubated at 37° C., 5% CO2, for 5 to (typically 10) minutes to facilitate cell attachment. After attachment, an initial imaging step was carried out using the RealTime-Glo™ MT assay from Promega (Catalogue No. G9711)) and the CellTox™ Green Cytotoxicity Assay (Promega, Catalogue No. G8741), carried out according to manufacturer's instructions. Following this initial imagining, the immunotherapeutic and/or chemotherapeutic agents were added to the cell populations as indicated below. After 10 minutes, the imagining step was repeated. The cell populations were then incubated at 37° C., 5% CO2 for 48 hours. The imaging step was then repeated. The cell populations were then re-dosed with the same immunotherapeutic and/or chemotherapeutic agents as indicated below, and incubated at 37° C., 5% CO2 for a further 48 hours. After this second 48 hour incubation, the imagining step was repeated for a final time.

At the end of this culture period, dose response scores were calculated using a normalized drug response metric as described in Gupta et al. (Communications Biology volume 3, Article number: 42 (2020), https://doi.org/10.1038/s42003-020-0765-z). The start readout for a given immunotherapeutic or chemotherapeutic agent at a given concentration was the to readout for said immunotherapeutic or chemotherapeutic agent at a specified concentration. The end readout for a given immunotherapeutic or chemotherapeutic agent at a given concentration was the final timepoint reading (t₉₆ₕ) for said immunotherapeutic or chemotherapeutic agent at a specified concentration.

The positive control start readout for a given drug/concentration was the to readout for the corresponding drug at a specified concentration. The positive control end readout for the corresponding drug at a specified concentration was the final timepoint reading (t₉₆ₕ) for the corresponding blank well.

The negative control start and end readouts for a given immunotherapeutic or chemotherapeutic agent at a given concentration were the to and final timepoint reading (t₉₆ₕ) respectively for blank cell or blank immuno cell well respectively.

The sensitivity or resistance of a subject's cancer to a given immunotherapeutic or chemotherapeutic agent was determined based on the calculated drug response score as shown in Table 2.

TABLE 2
Interpretation of drug response scores

| Drug Response Score | Interpretation |
|---|---|
| >1.0 | Resistant (−) |
| 0.0 to 1.0 | Effective (+) |
| −1.0 to 0.0 | Very Effective (++) |
| <−1.0 | Complete Clearance (+++) |

### Example 1-Determination of Treatment for Patient with Synovial Sarcoma

Blood samples from a patient with stage II synovial sarcoma (suspected spindle cell tumour) were received and tested using a 6 drug panel.

Numbers of CTC (EpCam+CD45⁻) were significantly higher than those observed in healthy volunteers, suggesting advanced cancer.

Chemotherapeutic agents were tested as in Table 3.

The drug response scores for this panel are shown in Table 4:

TABLE 4
Drug response scores for the 6 drug panel

| No. | Condition | conc. 1 | conc. 2 | conc. 3 | conc. 4 |
|---|---|---|---|---|---|
| 1 | Doxo | 0.9432 | 0.7234 | 0.8842 | 0.8405 |
| 2 | Ifos | 0.7534 | 0.6369 | 0.7555 | 0.5976 |
| 3 | Gemcit | 0.9382 | 0.4695 | 0.5149 | 0.4075 |
| 4 | Doce | 0.8633 | 0.7451 | 0.6992 | 0.4852 |
| 5 | Carbo | 1.4015 | 0.6497 | 0.4151 | 0.4317 |
| 6 | Dacarb | 2.1563 | 0.0887 | 0.1691 | 0.5700 |

Doxo = Doxorubicin;
Ifos = Ifosfamide;
Gemcit = Gemcitabine;
Doce = Docetaxel;
Carbo = Carboplatin;
Dacarb = Dacarbazine

Interpreting these drug response scores, the conclusions are shown in Table 5:

TABLE 5
Drug sensitivity as calculated by the assay
Based on Drug Response Scores the following interpretation is made:

| | | |
|---|---|---|
| 1 | Doxorubicin | Sensitive (S+) |
| 2 | Ifosfamide | Sensitive (S+) |
| 3 | Gemcitabine | Sensitive (S+) |
| 4 | Docetaxel | Sensitive (S+) |
| 5 | Carboplatin | Sensitive (S+) |
| 6 | Dacarbazine | Sensitive (S+) |

Under laboratory conditions, all drugs are effective (Sensitive S+) on Circulating Tumour Cells. Doxorubicin is marginally less effective compared to other drugs on Circulating Tumour Cells under laboratory conditions. Dacarbazine is marginally more effective compared to other drugs on Circulating Tumour Cells under laboratory conditions.

TABLE 3
Assay layout for Example 1

| | | Treatment | | | | | | | | | |
|---|---|---|---|---|---|---|---|---|---|---|---|
| | | | Blank | | | | | | | | |
| Row | | Blank | cells | Doxorubicin | | Ifosfamide | | | | Gemcitabine | |
| 1 | Drug | — | — | 10 ng/ml | 10 ng/ml | 0.125 | μg/ml | 0.125 | μg/ml | 2 μg/ml | 2 μg/ml |
| | concen- | | | | | | | | | | |
| | tration 1 | | | | | | | | | | |
| | Cells | — | CTCs | CTCs | CTCs | CTCs | | CTCs | | CTCS | CTCs |
| 2 | Drug | — | — | 30 ng/ml | 30 ng/ml | 0.25 | μg/ml | 0.25 | μg/ml | 10 μg/ml | 10 μg/ml |
| | concen- | | | | | | | | | | |
| | tration 2 | | | | | | | | | | |
| | Cells | — | CTCs | CTCs | CTCs | CTCs | | CTCs | | CTCs | CTCs |
| 3 | Drug | — | — | 90 ng/ml | 90 ng/ml | 5 | μg/ml | 5 | μg/ml | 20 μg/ml | 20 μg/ml |
| | concen- | | | | | | | | | | |
| | tration 3 | | | | | | | | | | |
| | Cells | — | CTCs | CTCs | CTCs | CTCs | | CTCs | | CTCs | CTCs |
| 4 | Drug | — | — | 270 ng/ml | 270 ng/ml | 10 | μg/ml | 10 | μg/ml | 40 μg/ml | 40 μg/ml |
| | concen- | | | | | | | | | | |
| | tration 4 | | | | | | | | | | |

| | | | | | | | | |
|---|---|---|---|---|---|---|---|---|
| Cells | — | CTCs | CTCs | CTCs | CTCs | CTCs | CTCs | CTCs |

| | | Treatment | | | | | | | | | |
|---|---|---|---|---|---|---|---|---|---|---|---|
| Row | | Docetaxel | | | | Carboplatin | | Dacarbazine | | | |
| 1 | Drug | 0.02 | μg/ml | 0.02 | μg/ml | 2 μg/ml | 2 μg/ml | 1.5 | μg/ml | 1.5 | μg/ml |
| | concen- | | | | | | | | | | |
| | tration 1 | | | | | | | | | | |
| | Cells | CTCs | | CTCs | | CTCs | CTCs | CTCs | | CTCs | |
| 2 | Drug | 0.2 | μg/ml | 0.2 | μg/ml | 5 μg/ml | 5 μg/ml | 3 | μg/ml | 3 | μg/ml |
| | concen- | | | | | | | | | | |
| | tration 2 | | | | | | | | | | |
| | Cells | CTCs | | CTCs | | CTCs | CTCs | CTCs | | CTCs | |
| 3 | Drug | 2 | μg/ml | 2 | μg/ml | 10 μg/ml | 10 μg/ml | 6 | μg/ml | 6 | μg/ml |
| | concen- | | | | | | | | | | |
| | tration 3 | | | | | | | | | | |
| | Cells | CTCs | | CTCs | | CTCs | CTCs | CTCs | | CTCs | |
| 4 | Drug | 10 | μg/ml | 10 | μg/ml | 20 μg/ml | 20 μg/ml | 12 | μg/ml | 12 | μg/ml |
| | concen- | | | | | | | | | | |
| | tration 4 | | | | | | | | | | |

| | | | | | | |
|---|---|---|---|---|---|---|
| Cells | CTCs | CTCs | CTCs | CTCs | CTCs | CTCs |

CTCs = circulating tumour cells

### Example 2-Determination of Treatment for Patient with Colon Cancer

Blood samples from a patient with colon cancer were received on 3 Mar. 2022 and tested using a 1 drug panel.

Numbers of CTC (EpCam+CD45⁻) were significantly higher than those observed in healthy volunteers, suggesting advanced cancer.

Chemotherapeutic agents were tested as in Table 6:

TABLE 6
Assay layout for Example 2

| | | Treatment | | | |
|---|---|---|---|---|---|
| | | | Blank | | |
| Row | | Blank | cells | Cisplatin | |
| 1 | Drug | — | — | 2 μg/ml | 2 μg/ml |
| | concentration 1 | | | | |
| | Cells | — | CTCs | CTCs | CTCs |
| 2 | Drug | — | — | 4 μg/ml | 4 μg/ml |
| | concentration 2 | | | | |
| | Cells | — | CTCs | CTCs | CTCs |
| 3 | Drug | — | — | 10 μg/ml | 10 μg/ml |
| | concentration 3 | | | | |
| | Cells | — | CTCs | CTCs | CTCs |
| 4 | Drug | — | — | 20 μg/ml | 20 μg/ml |
| | concentration 4 | | | | |
| | Cells | — | CTCs | CTCs | CTCs |

CTCs = circulating tumour cells

The drug response scores for this panel are shown in Table 7:

TABLE 7
Drug response scores for the 1 drug panel

| No. | Condition | conc. 1 | conc. 2 | conc. 3 | conc. 4 |
|---|---|---|---|---|---|
| 1 | Cis | 2.71948 | 2.873636 | 3.0122482 | 3.25869 |

Cis = Cisplatin

Interpreting these drug response scores, the conclusions are shown in Table 8:

TABLE 8
Drug sensitivity as calculated by the assay
Based on Drug Response Scores the following interpretation is made:

| | | |
|---|---|---|
| 1 | Cisplatin | Resistant |

Under laboratory conditions, Cisplatin is not effective on Circulating Tumour Cells.

### Example 3-Determination of Treatment for Patient with Rectal Cancer

Blood samples from a patient with rectal cancer (anorectal carcinoma with secondary lung cancer) were received on 17 Feb. 2022 and tested using a 4 drug panel.

Numbers of CTC (EpCam+CD45⁻) were significantly higher than those observed in healthy volunteers, suggesting advanced cancer.

Chemotherapeutic and immunotherapeutic agents were tested as in Table 9:

The drug response scores for this panel are shown in Table 10:

TABLE 10
Drug response scores for the 4 drug panel

| No. | Condition | conc. 1 | conc. 2 | conc. 3 | conc. 4 |
|---|---|---|---|---|---|
| 1 | Pembro | 0.6167 | 0.4053 | 0.9748 | 0.7712 |
| 2 | Nivo | 0.4990 | 0.6429 | 0.6261 | 1.0807 |
| 3 | Lenva | 2.0109 | 1.6409 | 0.8991 | 2.0235 |
| 4 | Regor | 0.9681 | 0.9212 | 0.7424 | −0.7024 |

Pembro = Pembrolizumab;
Nivo = Nivolumab;
Lenva = Lenvatinib;
Regor = Regorafinib

Interpreting these drug response scores, the conclusions are shown in Table 11:

TABLE 11
Drug sensitivity as calculated by the assay
Based on Drug Response Scores the following interpretation is made:

| | | |
|---|---|---|
| 1 | Pembrolizumab | Sensitive (S+) |
| 2 | Nivolumab | Sensitive (S+) |
| 3 | Lenvatinib | Resistant |
| 4 | Regorafinib | Sensitive (S+) |

Under laboratory conditions, Pembrolizumab, Nivolumab and Regorafinib are effective (Sensitive S+) on Circulating Tumour Cells. Under laboratory conditions Lenvatinib is not effective on Circulating Tumour Cells.

TABLE 9
Assay layout for Example 3

| | | Treatment | | | | | | | | | | |
|---|---|---|---|---|---|---|---|---|---|---|---|---|
| | | | Blank | | | | | | | | | |
| | | | immuno- | Blank | | | | | | | | |
| Row | | Blank | cells | cells | Pembrolizumab | | Nivolumab | | Lenvatinib | | Regorafinib | |
| 1 | Drug | — | — | — | 20 μg/ml | 20 μg/ml | 20 μg/ml | 20 μg/ml | 50 ng/ml | 50 ng/ml | 60 μg/ml | 60 μg/ml |
| | concen- | | | | | | | | | | | |
| | tration 1 | | | | | | | | | | | |
| | Cells | — | CTCs | CTCs | CTCs | CTCs | CTCs | CTCs | CTCs | CTCs | CTCs | CTCs |
| | | | CD45⁺ | | CD45⁺ | CD45⁺ | CD45⁺ | CD45⁺ | | | | |
| | | | cells | | cells | cells | cells | cells | | | | |
| 2 | Drug | — | — | — | 40 μg/ml | 40 μg/ml | 40 μg/ml | 40 μg/ml | 100 ng/ml | 100 ng/ml | 80 μg/ml | 80 μg/ml |
| | concen- | | | | | | | | | | | |
| | tration 2 | | | | | | | | | | | |
| | Cells | — | CTCs | CTCs | CTCs | CTCs | CTCs | CTCs | CTCs | CTCs | CTCs | CTCs |
| | | | CD45⁺ | | CD45⁺ | CD45⁺ | CD45⁺ | CD45⁺ | | | | |
| | | | cells | | cells | cells | cells | cells | | | | |
| 3 | Drug | — | — | — | 100 μg/ml | 100 μg/ml | 80 μg/ml | 80 μg/ml | 200 ng/ml | 200 ng/ml | 120 μg/ml | 120 μg/ml |
| | concen- | | | | | | | | | | | |
| | tration 3 | | | | | | | | | | | |
| | Cells | — | CTCs | CTCs | CTCs | CTCs | CTCs | CTCs | CTCs | CTCs | CTCs | CTCs |
| | | | CD45⁺ | | CD45⁺ | CD45⁺ | CD45⁺ | CD45⁺ | | | | |
| | | | cells | | cells | cells | cells | cells | | | | |
| 4 | Drug | — | — | — | 160 μg/ml | 160 μg/ml | 160 μg/ml | 160 μg/ml | 400 ng/ml | 400 ng/ml | 160 μg/ml | 160 μg/ml |
| | concen- | | | | | | | | | | | |
| | tration 4 | | | | | | | | | | | |
| | Cells | — | CTCs | CTCs | CTCs | CTCs | CTCs | CTCs | CTCs | CTCs | CTCs | CTCs |
| | | | CD45⁺ | | CD45⁺ | CD45⁺ | CD45⁺ | CD45⁺ | | | | |
| | | | cells | | cells | cells | cells | cells | | | | |

CTCs = circulating tumour cells;
CD45⁺ = CD45⁺ cells

### Example 4-Determination of Treatment for Patient with Sex Cord Stromal Tumour, Ovarian Tumour

Blood samples from a patient with sex cord stromal tumour, ovarian tumour (epitheliod neoplasm, stage 3 recurrence after surgery (resection) and previous chemotherapy) were received on 19 Jul. 2021 and tested using a 5 drug panel.

Numbers of CTC (EpCam+CD45⁻) were significantly higher than those observed in healthy volunteers, suggesting advanced cancer.

Chemotherapeutic agents were tested as in Table 12:

The drug response scores for this panel are shown in Table 13:

TABLE 13
Drug response scores for the 5 drug panel

| No. | Condition | conc. 1 | conc. 2 | conc. 3 | conc. 4 |
|---|---|---|---|---|---|
| 1 | Cyclo | 0.97 | 0.58 | 0.41 | 0.94 |
| 2 | Vinc | 3.35 | 1.24 | 1.83 | 1.29 |
| 3 | Etop | 0.79 | 1.06 | 1.19 | 1.35 |
| 4 | Bini | 0.93 | 1.18 | 1.19 | 1.06 |
| 5 | Doxo | 0.68 | 0.93 | 0.54 | 0.93 |

Cyclo = Cyclophosphamide;
Vinc = Vincristine;
Etop = Etoposide;
Bini = Binimetinib;
Doxo = Doxorubicin (Adriamycin)

Interpreting these drug response scores, the conclusions are shown in Table 14:

TABLE 14
Drug sensitivity as calculated by the assay
Based on Drug Response Scores the following interpretation is made:

| | | |
|---|---|---|
| 1 | Cyclophosphamide | Sensitive (S++) |
| 2 | Vincristine | Resistant |
| 3 | Etoposide | Resistant |
| 4 | Binimetinib | Resistant |
| 5 | Doxorubicin (Adriamycin) | Sensitive (S++) |

Under laboratory conditions, Cyclophosphamide and Adriamycin (Doxorubicin) show effectiveness on Circulating Tumour Cells (CTCs). Vincristine, Etoposide and Binimetinib are not effective under laboratory conditions.

TABLE 12
Assay layout for Example 4

| | | | | Blank | | | | | | | | |
|---|---|---|---|---|---|---|---|---|---|---|---|---|
| Row | | Blank | | cells | | Cyclophosphamide | | | | Vincristine | | Etoposide |
| 1 | Drug | — | — | — | — | 2.5 | μg/ml | 2.5 | μg/ml | 10 ng/ml | 10 ng/ml | 1 μg/ml |
| | concen- | | | | | | | | | | | |
| | tration 1 | | | | | | | | | | | |
| | Cells | — | — | CTCs | CTCs | CTCs | | CTCs | | CTCs | CTCs | CTCs |
| 2 | Drug | — | — | — | — | 10 | μg/ml | 10 | μg/ml | 20 ng/ml | 20 ng/ml | 4 μg/ml |
| | concen- | | | | | | | | | | | |
| | tration 2 | | | | | | | | | | | |
| | Cells | — | — | CTCs | CTCs | CTCs | | CTCs | | CTCs | CTCs | CTCs |
| 3 | Drug | — | — | — | — | 40 | μg/ml | 40 | μg/ml | 40 ng/ml | 40 ng/ml | 20 μg/ml |
| | concen- | | | | | | | | | | | |
| | tration 3 | | | | | | | | | | | |
| | Cells | — | — | CTCs | CTCs | CTCs | | CTCs | | CTCs | CTCs | CTCs |
| 4 | Drug | — | — | — | — | 160 | μg/ml | 160 | μg/ml | 80 ng/ml | 80 ng/ml | 100 μg/ml |
| | concen- | | | | | | | | | | | |
| | tration 4 | | | | | | | | | | | |
| | Cells | — | — | CTCs | CTCs | CTCs | | CTCs | | CTCs | CTCs | CTCs |

| Row | | Etoposide | Binimetinib | | Doxorubicin | |
|---|---|---|---|---|---|---|
| 1 | Drug | 1 μg/ml | 100 ng/ml | 100 ng/ml | 10 ng/ml | 10 ng/ml |
| | concen- | | | | | |
| | tration 1 | | | | | |
| | Cells | CTCs | CTCs | CTCs | CTCs | CTCs |
| 2 | Drug | 4 μg/ml | 200 ng/ml | 200 ng/ml | 30 ng/ml | 30 ng/ml |
| | concen- | | | | | |
| | tration 2 | | | | | |
| | Cells | CTCs | CTCs | CTCs | CTCs | CTCs |
| 3 | Drug | 20 μg/ml | 400 ng/ml | 400 ng/ml | 90 ng/ml | 90 ng/ml |
| | concen- | | | | | |
| | tration 3 | | | | | |
| | Cells | CTCs | CTCs | CTCs | CTCs | CTCs |
| 4 | Drug | 100 μg/ml | 800 ng/ml | 800 ng/ml | 270 ng/ml | 270 ng/ml |
| | concen- | | | | | |
| | tration 4 | | | | | |
| | Cells | CTCs | CTCs | CTCs | CTCs | CTCs |

CTCs = circulating tumour cells

### Example 5-Determination of Treatment for Patient with Lung Cancer

Blood samples from a patient with lung cancer (adenocarcinoma, progression after first-line chemotherapy) were received on 9 Jul. 2021 and tested using a 10 drug panel. Numbers of CTC (EpCam+CD45⁻) were significantly higher than those observed in healthy volunteers, suggesting advanced cancer.

Chemotherapeutic and immunotherapeutic agents were tested as in Table 15: The drug response scores for this panel are shown in Table 16:

TABLE 16
Drug response scores for the 10 drug panel

| No. | Condition | conc. 1 | conc. 2 | conc. 3 | conc. 4 |
|---|---|---|---|---|---|
| 1 | Niv | −0.45 | −0.80 | −0.12 | −0.22 |
| 2 | Pem | −0.34 | −0.15 | 0.05 | 0.24 |
| 3 | Gm | −0.62 | −0.48 | 0.22 | 0.54 |
| 4 | Vin | 0.34 | −0.32 | 0.37 | −0.16 |
| 5 | Gf | 0.70 | 0.22 | 0.30 | 0.07 |
| 6 | E | 0.03 | −0.20 | 0.13 | 0.42 |
| 7 | Afa | 0.48 | −0.20 | 0.00 | 0.32 |
| 8 | Metho | −0.43 | −0.74 | 0.05 | 0.04 |
| 9 | Cz | −0.59 | 0.16 | −0.75 | 0.11 |
| 10 | Pal | 0.21 | −0.03 | −0.41 | 0.16 |

Niv = Nivolumab;
Pem = Pembrozulimab;
Gm = Gemcitabine;
Vin = Vinorelbine;
Gf = Gefitinib;
E = Erlotinib;
Afa = Afatinib;
Metho = Methotrexate;
Cz = Crizotinib;
Pal = Palbociclib

Interpreting these drug response scores, the conclusions are shown in Table 17:

TABLE 17
Drug sensitivity as calculated by the assay
Based on Drug Response Scores the following interpretation is made:

| | | |
|---|---|---|
| 1 | Nivolumab | S++ |
| 2 | Pembrozulimab | S++ |
| 3 | Gemcitabine | S++ |
| 4 | Vinorelbine | S++ |
| 5 | Gefitinib | S+ |
| 6 | Erlotinib | S+ |
| 7 | Afatinib | S+ |
| 8 | Methotrexate | S++ |
| 9 | Crizotinib | S++ |
| 10 | Palbociclib | S++ |

Under laboratory conditions, all drugs show effectiveness on Circulating Tumour Cells (CTCs). Gefitinib, Erlotinib and Afatinib are relatively less effective compared rest of the agents. Amongst Immunotherapy agents, Nivolumab is more effective than Pembrozulimab. Amongst other agents, Gemcitabine and Crizotinib are marginally more effective than other agents.

TABLE 15
Assay layout for Example 5

| | | Treatment | | | | | | |
|---|---|---|---|---|---|---|---|---|
| | | | Blank | | | | | |
| | | | immuno- | Blank | | | | |
| Row | | Blank | cells | cells | Nivolumab | Pembrozulimab | Gemcitabine | Vinorelbine |
| 1 | Drug | — | — | — | 20 μg/ml | 20 μg/ml | 2 μg/ml | 20 μg/ml |
| | conc 1 | | | | | | | |
| | Cells | — | CTCs | CTCs | CTCs | CTCs | CTCs | CTCs |
| | | | CD45⁺ | | CD45⁺ | CD45⁺ | | |
| | | | cells | | cells | cells | | |
| 2 | Drug | — | — | — | 20 μg/ml | 20 μg/ml | 10 μg/ml | 40 μg/ml |
| | conc 2 | | | | | | | |
| | Cells | — | CTCs | CTCs | CTCs | CTCs | CTCs | CTCs |
| | | | CD45⁺ | | CD45⁺ | CD45⁺ | | |
| | | | cells | | cells | cells | | |
| 3 | Drug | — | — | — | 80 μg/ml | 100 μg/ml | 20 μg/ml | 80 μg/ml |
| | conc 3 | | | | | | | |
| | Cells | — | CTCs | CTCs | CTCs | CTCs | CTCs | CTCs |
| | | | CD45⁺ | | CD45⁺ | CD45⁺ | | |
| | | | cells | | cells | cells | | |
| 4 | Drug | — | — | — | 160 μg/ml | 160 μg/ml | 40 μg/ml | 200 μg/ml |
| | conc 4 | | | | | | | |
| | Cells | — | CTCs | CTCs | CTCs | CTCs | CTCs | CTCs |
| | | | CD45⁺ | | CD45⁺ | CD45⁺ | | |
| | | | cells | | cells | cells | | |

| | | Treatment | | | | | | | |
|---|---|---|---|---|---|---|---|---|---|
| | Row | Gefitinib | Erlotinib | | Afatinib | Methotrexate | | Crizotinib | Palbociclib |
| 1 | Drug | 100 ng/ml | 0.75 | μg/ml | 20 ng/ml | 0.5 | μg/ml | 200 ng/ml | 40 ng/ml |
| | conc 1 | | | | | | | | |
| | Cells | CTCs | CTCs | | CTCs | CTCs | | CTCs | CTCs |
| 2 | Drug | 200 ng/ml | 1.5 | μg/ml | 50 ng/ml | 1 | μg/ml | 1000 ng/ml | 80 ng/ml |
| | conc 2 | | | | | | | | |
| | Cells | CTCs | CTCs | | CTCs | CTCs | | CTCs | CTCs |
| 3 | Drug | 600 ng/ml | 3 | μg/ml | 100 ng/ml | 2 | μg/ml | 2000 ng/ml | 160 ng/ml |
| | conc 3 | | | | | | | | |
| | Cells | CTCs | CTCs | | CTCs | CTCs | | CTCs | CTCs |
| 4 | Drug | 1200 ng/ml | 6 | μg/ml | 150 ng/ml | 4 | μg/ml | 4000 ng/ml | 320 ng/ml |
| | conc 4 | | | | | | | | |

| | | | | | | |
|---|---|---|---|---|---|---|
| Cells | CTCs | CTCs | CTCs | CTCs | CTCs | CTCs |

CTCs = circulating tumour cells;
CD45⁺ = CD45⁺ cells

### Example 6-Determination of Treatment for Patient with Head and Neck Cancer

Blood samples from a patient with head and neck cancer (squamous cell carcinoma) were received on 11 Mar. 2022 and tested using a 7 drug panel.

Numbers of CTC (EpCam+CD45) were significantly higher than those observed in healthy volunteers, suggesting advanced cancer.

Chemotherapeutic agents were tested as in Table 18: The drug response scores for this panel are shown in Table 19:

TABLE 19
Drug response scores for the 7 drug panel

| No. | Condition | conc. 1 | conc. 2 | conc. 3 | conc. 4 |
|---|---|---|---|---|---|
| 1 | Ifos | 0.7257 | 0.2439 | 0.9228 | 0.3682 |
| 2 | Gemcit | 0.9091 | 0.8443 | 0.6925 | 0.3157 |
| 3 | Doxo | 0.7699 | 0.9452 | 0.6229 | 0.7995 |
| 4 | Etop | 0.9372 | 0.6295 | 0.9862 | 0.5135 |
| 5 | Metho | 0.9711 | 0.9988 | 1.0559 | 0.7375 |
| 6 | Capecit | 0.7820 | 0.9244 | 0.6113 | 1.0037 |
| 7 | Vinor | 0.6102 | 0.9691 | 0.4733 | 0.8073 |

Ifos = Ifosfamide;
Gemcit = Gemcitabine;
Doxo = Doxorubicin;
Etop = Etoposide;
Metho = Methotrexate;
Capecit = Capecitabine;
Vinor = Vinorelbine

Interpreting these drug response scores, the conclusions are shown in Table 20:

TABLE 20
Drug sensitivity as calculated by the assay
Based on Drug Response Scores the following interpretation is made:

| | | |
|---|---|---|
| 1 | Ifosfamide | Sensitive (S+) |
| 2 | Gemcitabine | Sensitive (S+) |
| 3 | Doxorubicin | Sensitive (S+) |
| 4 | Etoposide | Sensitive (S+) |
| 5 | Methotrexate | Resistant |
| 6 | Capecitabine | Resistant |
| 7 | Vinorelbine | Sensitive (S+) |

Under laboratory conditions Ifosfamide, Gemcitabine, Doxorubicin, Etoeoside and Vinorelbine are effective (Sensitive S+) on Circulating Tumour Cells. Methotrexate and Capecitabine are not effective on the Circulating Tumour Cells under laboratory conditions.

TABLE 18
Assay layout for Example 6

| | | Treatment | | | | | | | | | | |
|---|---|---|---|---|---|---|---|---|---|---|---|---|
| | | | Blank | | | | | | | | | |
| Row | | Blank | cells | Ifosfamide | | | | Gemcitabine | | Doxorubicin | | Etoposide |
| 1 | Drug | — | — | 0.125 | μg/ml | 0.125 | μg/ml | 2 μg/ml | 2 μg/ml | 10 ng/ml | 10 ng/ml | 1 μg/ml |
| | conc 1 | | | | | | | | | | | |
| | Cells | — | CTCs | CTCs | | CTCs | | CTCs | CTCs | CTCs | CTCs | CTCs |
| 2 | Drug | — | — | 0.25 | μg/ml | 0.25 | μg/ml | 10 μg/ml | 10 μg/ml | 30 ng/ml | 30 ng/ml | 4 μg/ml |
| | conc 2 | | | | | | | | | | | |
| | Cells | — | CTCs | CTCs | | CTCs | | CTCs | CTCs | CTCs | CTCs | CTCs |
| 3 | Drug | — | — | 5 | μg/ml | 5 | μg/ml | 20 μg/ml | 20 μg/ml | 90 ng/ml | 90 ng/ml | 20 μg/ml |
| | conc 3 | | | | | | | | | | | |
| | Cells | — | CTCs | CTCs | | CTCs | | CTCs | CTCs | CTCs | CTCs | CTCs |
| 4 | Drug | — | — | 10 | μg/ml | 10 | μg/ml | 40 μg/ml | 40 μg/ml | 270 ng/ml | 270 ng/ml | 100 μg/ml |
| | conc 4 | | | | | | | | | | | |

| | | | | | | | | | |
|---|---|---|---|---|---|---|---|---|---|
| Cells | — | CTCs | CTCs | CTCs | CTCs | CTCs | CTCs | CTCs | CTCs |

| | | Treatment | | | | | | | | |
|---|---|---|---|---|---|---|---|---|---|---|
| Row | | Etoposide | Methotrexate | | | | Capecitabine | | Vinorelbine | |
| 1 | Drug | 1 μg/ml | 0.5 | μg/ml | 0.5 | μg/ml | 200 μg/ml | 200 μg/ml | 20 μg/ml | 20 μg/ml |
| | conc 1 | | | | | | | | | |
| | Cells | CTCs | CTCs | | CTCs | | CTCs | CTCs | CTCs | CTCs |
| 2 | Drug | 4 μg/ml | 1 | μg/ml | 1 | μg/ml | 300 μg/ml | 300 μg/ml | 40 μg/ml | 40 μg/ml |
| | conc 2 | | | | | | | | | |
| | Cells | CTCs | CTCs | | CTCs | | CTCs | CTCs | CTCs | CTCs |
| 3 | Drug | 20 μg/ml | 2 | μg/ml | 2 | μg/ml | 400 μg/ml | 400 μg/ml | 80 μg/ml | 80 μg/ml |
| | conc 3 | | | | | | | | | |
| | Cells | CTCs | CTCs | | CTCs | | CTCs | CTCs | CTCs | CTCs |
| 4 | Drug | 100 μg/ml | 4 | μg/ml | 4 | μg/ml | 600 μg/ml | 600 μg/ml | 200 μg/ml | 200 μg/ml |
| | conc 4 | | | | | | | | | |

| | | | | | | | |
|---|---|---|---|---|---|---|---|
| Cells | CTCs | CTCs | CTCs | CTCs | CTCs | CTCs | CTCs |

CTCs = circulating tumour cells

### Example 7-Determination of Treatment for Patient with Renal Cancer

Blood samples from a patient with renal cancer were received on 9 Mar. 2022 and tested using a 6 drug panel.

Numbers of CTC (EpCam+CD45⁻) were significantly higher than those observed in healthy volunteers, suggesting advanced cancer.

Chemotherapeutic and immunotherapeutic agents were tested as in Table 21: The drug response scores for this panel are shown in Table 22:

TABLE 22
Drug response scores for the 6 drug panel

| No. | Condition | conc. 1 | conc. 2 | conc. 3 | conc. 4 |
|---|---|---|---|---|---|
| 1 | Gemcit | −0.1637 | 0.0725 | −0.3154 | −0.0871 |
| 2 | Docetax | 0.0585 | −0.1811 | 0.0514 | −0.1468 |
| 3 | Topo | 0.1847 | −0.0648 | −0.0964 | −0.1707 |
| 4 | Etop | −0.2179 | −0.1141 | −0.1357 | −0.0050 |
| 5 | Eribul | 0.4482 | 0.0692 | −0.1453 | −0.0193 |
| 10 | Pembr | −0.0194 | 0.1577 | 0.0468 | −0.0364 |

Gemcit = Gemcitabine;
Docetax = Docetaxel;
Topo = Topotecan;
Etop = Etoposide;
Eribul = Eribuline;
Pembr = Pembrolizumab

Interpreting these drug response scores, the conclusions are shown in Table 23:

TABLE 23
Drug sensitivity as calculated by the assay
Based on Drug Response Scores the following interpretation is made:

| | | |
|---|---|---|
| 1 | Gemcitabine | Sensitive (S++) |
| 2 | Docetaxel | Sensitive (S+) |
| 3 | Topotecan | Sensitive (S++) |
| 4 | Etoposide | Sensitive (S++) |
| 5 | Eribuline | Sensitive (S+) |
| 6 | Pembrolizumab | Sensitive (S+) |

Under laboratory conditions, Pembrolizumab (immunotherapy) is effective (Sensitive S+) on Circulating Tumour Cells. Under laboratory conditions, all drugs show effectiveness on Circulating Tumour Cells with Gemcitabine, Topotecan and Etoposide more effective (Sensitive S++)

TABLE 21
Assay layout for Example 7

| | | Treatment | | | | | | | | | | |
|---|---|---|---|---|---|---|---|---|---|---|---|---|
| | | | Blank | | | | | | | | | |
| | | | immuno- | Blank | | | | | | | | |
| Row | | Blank | cells | cells | Pembrozulimab | | Docetaxel | | | | Gemcitabine | |
| 1 | Drug | — | — | — | 20 μg/ml | 20 μg/ml | 0.02 | μg/ml | 0.02 | μg/ml | 2 μg/ml | 2 μg/ml |
| | conc 1 | | | | | | | | | | | |
| | Cells | — | CTCs | CTCs | CTCs | CTCs | CTCs | | CTCs | | CTCs | CTCs |
| | | | CD45⁺ | | CD45⁺ | CD45⁺ | | | | | | |
| | | | cells | | cells | cells | | | | | | |
| 2 | Drug | — | — | — | 40 μg/ml | 40 μg/ml | 0.2 | μg/ml | 0.2 | μg/ml | 10 μg/ml | 10 μg/ml |
| | conc 2 | | | | | | | | | | | |
| | Cells | — | CTCs | CTCs | CTCs | CTCs | CTCs | | CTCs | | CTCs | CTCs |
| | | | CD45⁺ | | CD45⁺ | CD45⁺ | | | | | | |
| | | | cells | | cells | cells | | | | | | |
| 3 | Drug | — | — | — | 100 μg/ml | 100 μg/ml | 2 | μg/ml | 2 | μg/ml | 20 μg/ml | 20 μg/ml |
| | conc 3 | | | | | | | | | | | |
| | Cells | — | CTCs | CTCs | CTCs | CTCs | CTCs | | CTCs | | CTCs | CTCs |
| | | | CD45⁺ | | CD45⁺ | CD45⁺ | | | | | | |
| | | | cells | | cells | cells | | | | | | |
| 4 | Drug | — | — | — | 160 μg/ml | 160 μg/ml | 10 | μg/ml | 10 | μg/ml | 40 μg/ml | 40 μg/ml |
| | conc 4 | | | | | | | | | | | |

| | | | | | | | | | |
|---|---|---|---|---|---|---|---|---|---|
| Cells | — | CTCs | CTCs | CTCs | CTCs | CTCs | CTCs | CTCs | CTCs |
| | | CD45⁺ | | CD45⁺ | CD45⁺ | | | | |
| | | cells | | cells | cells | | | | |

| | | Treatment | | | | | |
|---|---|---|---|---|---|---|---|
| Row | | Topotecan | | Etoposide | | Eribuline | |
| 1 | Drug | 5 μg/ml | 5 μg/ml | 1 μg/ml | 1 μg/ml | 5 μg/ml | 5 μg/ml |
| | conc 1 | | | | | | |
| | Cells | CTCs | CTCs | CTCs | CTCs | CTCs | CTCs |
| 2 | Drug | 10 μg/ml | 10 μg/ml | 4 μg/ml | 4 μg/ml | 10 μg/ml | 10 μg/ml |
| | conc 2 | | | | | | |
| | Cells | CTCs | CTCs | CTCs | CTCs | CTCs | CTCs |
| 3 | Drug | 50 μg/ml | 50 μg/ml | 20 μg/ml | 20 μg/ml | 20 μg/ml | 20 μg/ml |
| | conc 3 | | | | | | |
| | Cells | CTCs | CTCs | CTCs | CTCs | CTCs | CTCs |
| 4 | Drug | 100 μg/ml | 100 μg/ml | 100 μg/ml | 100 μg/ml | 40 μg/ml | 40 μg/ml |
| | conc 4 | | | | | | |
| | Cells | CTCs | CTCs | CTCs | CTCs | CTCs | CTCs |

CTCs = circulating tumour cells;
CD45⁺ = CD45⁺ cells

### Example 8-Determination of Treatment for Patient with Colon Cancer

Blood samples from a patient with colon cancer (sigmoid colon cancer) were received on 8 Mar. 2022 and tested using a 9 drug panel.

Numbers of CTC (EpCam+CD45⁻) were significantly higher than those observed in healthy volunteers, suggesting advanced cancer.

Chemotherapeutic agents were tested as in Table 24.

The drug response scores for this panel are shown in Table 25:

TABLE 25
Drug response scores for the 9 drug panel

| No. | Condition | conc. 1 | conc. 2 | conc. 3 | conc. 4 |
|---|---|---|---|---|---|
| 1 | Eribul | 4.4627 | −0.1620 | −0.0329 | −0.4785 |
| 2 | Pacli | −0.8976 | −0.1500 | −0.5818 | −0.8009 |
| 3 | Vinor | 0.0721 | −0.6648 | 0.2340 | −0.5738 |
| 4 | Dox | −0.0102 | −0.0852 | −0.2828 | −0.0485 |
| 5 | Cyclophos | −0.3176 | 0.9581 | 0.6810 | 2.5058 |
| 6 | Etoposide | −0.4099 | −0.6338 | −0.6648 | 0.1129 |
| 7 | Temozol | −1.0000 | 0.0367 | −0.1421 | −0.4687 |
| 8 | Carbo | 6.6672 | −0.0506 | −0.8131 | −0.1322 |
| 9 | Pemet | 5.1780 | −1.0000 | −1.0000 | −1.0000 |

Eribul = Eribuline;
Pacli = Paclitaxel;
Vinor = Vinorelbine;
Dox = Doxorubicin;
Cyclophos = Cyclophosphamide;
Etop = Etoposide;
Temozol = Temozolamide;
Carbo = Carboplatin;
Pemet = Pemetrexed

Interpreting these drug response scores, the conclusions are shown in Table 26:

TABLE 26
Drug sensitivity as calculated by the assay
Based on Drug Response Scores the following interpretation is made:

| | | |
|---|---|---|
| 1 | Eribuline | Sensitive (S++) |
| 2 | Paclitaxel | Sensitive (S++) |
| 3 | Vinorelbine | Sensitive (S+) |
| 4 | Doxorubicin | Sensitive (S++) |
| 5 | Cyclophosphamide | Sensitive (S+) |
| 6 | Etoposide | Sensitive (S++) |
| 7 | Temozolamide | Sensitive (S++) |
| 8 | Carboplatin | Sensitive (S++) |
| 9 | Pemetrexed | Sensitive (S+++) |

All drugs are effective on Circulating Tumour Cells under laboratory conditions with Pemetrexed is most effective (S+++} and Cyclophosphamide is Least effective (S+).

TABLE 24
Assay layout for Example 8

| | | Treatment | | | | | | | | | | | | |
|---|---|---|---|---|---|---|---|---|---|---|---|---|---|---|
| | | | Blank | | | | | | Cyclophos- | | | | | |
| Row | | Blank | cells | Eribuline | Paclitaxel | | Vinorelbine | Doxorubicin | phamide | | Temozolamide | | Carboplatin | Pemetrexed |
| 1 | Drug | — | — | 5 μg/ml | 0.05 | μg/ml | 20 μg/ml | 10 ng/ml | 2.5 | μg/ml | 0.5 | μg/ml | 2 μg/ml | 22 μg/ml |
| | conc 1 | | | | | | | | | | | | | |
| | Cells | — | CTCs | CTCs | CTCs | | CTCs | CTCs | CTCs | | CTCs | | CTCs | CTCs |
| 2 | Drug | — | — | 10 μg/ml | 0.1 | μg/ml | 40 μg/ml | 30 ng/ml | 10 | μg/ml | 1 | μg/ml | 5 μg/ml | 110 μg/ml |
| | conc 2 | | | | | | | | | | | | | |
| | Cells | — | CTCs | CTCs | CTCs | | CTCs | CTCs | CTCs | | CTCs | | CTCs | CTCs |
| 3 | Drug | — | — | 20 μg/ml | 0.2 | μg/ml | 80 μg/ml | 90 ng/ml | 40 | μg/ml | 2 | μg/ml | 10 μg/ml | 220 μg/ml |
| | conc 3 | | | | | | | | | | | | | |
| | Cells | — | CTCs | CTCs | CTCs | | CTCs | CTCs | CTCs | | CTCs | | CTCs | CTCs |
| 4 | Drug | — | — | 40 μg/ml | 1 | μg/ml | 200 μg/ml | 270 ng/ml | 160 | μg/ml | 4 | μg/ml | 20 μg/ml | 440 μg/ml |
| | conc 4 | | | | | | | | | | | | | |

| | | | | | | | | | | |
|---|---|---|---|---|---|---|---|---|---|---|
| Cells | — | CTCs | CTCs | CTCs | CTCs | CTCs | CTCs | CTCs | CTCs | CTCs |

CTCs = circulating tumour cells.

### Example 9-Determination of Treatment for Patient with GI Cancer

Blood and tissue samples from a patient with GI cancer (Cholangio Carcinoma, stage 4, from a patient previously given cisplatin and gemcitabine) were received on 4 Feb. 2022 and tested using a 8 drug panel.

Numbers of CTC (EpCam+CD45) were significantly higher than those observed in healthy volunteers, suggesting advanced cancer.

Chemotherapeutic and immunotherapeutic agents were tested as in Table 27A and B.

The drug response scores for this panel are shown in Tables 28 and 29:

TABLE 28
Drug response scores for the 8 drug panel from blood sample

| No. | Condition | conc. 1 | conc. 2 | conc. 3 | conc. 4 |
|---|---|---|---|---|---|
| 1 | Pem | 0.00068 | 0.15720 | 1.04116 | 0.69890 |
| 2 | Niv | 1.00000 | 0.27831 | 1.06122 | 1.04569 |
| 3 | Atez | 0.10368 | 0.14987 | 0.34935 | 0.43914 |
| 4 | Len | 0.28509 | 0.31546 | 0.40967 | 0.62138 |
| 5 | Pacli | 0.23462 | 0.53292 | 0.52981 | 0.75597 |
| 6 | Irino | 0.19738 | 0.34688 | 0.61973 | 0.59130 |
| 7 | 5-FU | 0.41468 | 0.34101 | 0.42204 | 0.08131 |
| 8 | Beva | 0.46054 | 1.25685 | 1.83703 | 2.20970 |

Pem = Pembrolizumab;
Niv = Nivolumab;
Atez = Atezolizumab;
Len = Lenvatinib;
Pacli = Paclitaxel;
Irino = Irinotecan;
5-FU = 5 FluoroUracil;
Beva = Bevacizumab

TABLE 29
Drug response scores for the 8 drug panel from tissue sample

| No. | Condition | conc. 1 | conc. 2 | conc. 3 | conc. 4 |
|---|---|---|---|---|---|
| 1 | Pem | 0.97641 | 0.9717059 | 0.9671627 | 0.83541 |
| 2 | Niv | 0.94244 | 0.9636013 | 0.9017727 | 0.68327 |
| 3 | Atez | 0.89043 | 0.9046632 | 0.8647177 | 0.79535 |
| 4 | Len | 0.90822 | 0.9131589 | 0.8739489 | 0.94335 |
| 5 | Pacli | 0.93652 | 0.9526443 | 0.9071675 | 0.96833 |
| 6 | Irino | 0.88698 | 0.9134747 | 0.8741725 | 0.86121 |
| 7 | 5-FU | 0.93342 | 0.9487939 | 0.9923641 | 0.01222 |
| 8 | Beva | 0.93004 | 0.9294091 | 0.9320316 | 0.92612 |

Pem = Pembrolizumab;
Niv = Nivolumab;
Atez = Atezolizumab;
Len = Lenvatinib;
Pacli = Paclitaxel;
Irino = Irinotecan;
5-FU = 5 FluoroUracil;
Beva = Bevacizumab

TABLE 27

| | Treatment | | | | | | |
|---|---|---|---|---|---|---|---|
| | | | Blank | | | | |
| | | Blank | immuno- | Pembro- | | Atezo- | |
| Row | Blank | cells | cells | lizumab | Nivolumab | lizumab | Lenvatinib |

A: Assay layout for Example 9 - blood sample

| | | | | | | | | | | | | |
|---|---|---|---|---|---|---|---|---|---|---|---|---|
| 1 | Drug | — | — | — | 20 | 20 | 20 | 20 | 100 | 100 | 50 | 50 |
| | conc | | | | μg/ml | μg/ml | μg/ml | μg/ml | μg/ml | μg/ml | ng/ml | ng/ml |
| | 1 | | | | | | | | | | | |
| | Cells | — | CTCs | CTCs | CTCs | CTCs | CTCs | CTCs | CTCs | CTCs | CTCs | CTCs |
| | | | T −ve | CD45⁺ | CD45⁺ | CD45⁺ | CD45⁺ | CD45⁺ | CD45⁺ | CD45⁺ | T −ve | T −ve |
| | | | | T −ve | T −ve | T −ve | T −ve | T −ve | T −ve | T −ve | | |
| 2 | Drug | — | — | — | 40 | 40 | 40 | 40 | 200 | 200 | 100 | 100 |
| | conc | | | | μg/ml | μg/ml | μg/ml | μg/ml | μg/ml | μg/ml | ng/ml | ng/ml |
| | 2 | | | | | | | | | | | |
| | Cells | — | CTCs | CTCs | CTCs | CTCs | CTCs | CTCs | CTCs | CTCs | CTCs | CTCs |
| | | | T −ve | CD45⁺ | CD45⁺ | CD45⁺ | CD45⁺ | CD45⁺ | CD45⁺ | CD45⁺ | T −ve | T −ve |
| | | | | T −ve | T −ve | T −ve | T −ve | T −ve | T −ve | T −ve | | |
| 3 | Drug | — | — | — | 100 | 100 | 80 | 80 | 400 | 400 | 200 | 200 |
| | conc | | | | μg/ml | μg/ml | μg/ml | μg/ml | μg/ml | μg/ml | ng/ml | ng/ml |
| | 3 | | | | | | | | | | | |
| | Cells | — | CTCs | CTCs | CTCs | CTCs | CTCs | CTCs | CTCs | CTCs | CTCs | CTCs |
| | | | T −ve | CD45⁺ | CD45⁺ | CD45⁺ | CD45⁺ | CD45⁺ | CD45⁺ | CD45⁺ | T −ve | T −ve |
| | | | | T −ve | T −ve | T −ve | T −ve | T −ve | T −ve | T −ve | | |
| 4 | Drug | — | — | — | 160 | 160 | 160 | 160 | 800 | 800 | 400 | 400 |
| | conc | | | | μg/ml | μg/ml | μg/ml | μg/ml | μg/ml | μg/ml | ng/ml | ng/ml |
| | 4 | | | | | | | | | | | |
| | Cells | — | CTCs | CTCs | CTCs | CTCs | CTCs | CTCs | CTCs | CTCs | CTCs | CTCs |
| | | | T −ve | CD45⁺ | CD45⁺ | CD45⁺ | CD45⁺ | CD45⁺ | CD45⁺ | CD45⁺ | T −ve | T −ve |
| | | | | T −ve | T −ve | T −ve | T −ve | T −ve | T −ve | T −ve | | |

B: Assay layout for Example 9 - tissue sample

| | | | | | | | | | | | | |
|---|---|---|---|---|---|---|---|---|---|---|---|---|
| 1 | Drug | — | — | — | 20 | 20 | 20 | 20 | 100 | 100 | 50 | 50 |
| | conc | | | | μg/ml | μg/ml | μg/ml | μg/ml | μg/ml | μg/ml | ng/ml | ng/ml |
| | 1 | | | | | | | | | | | |
| | Cells | — | CTCs | CTCs | CTCs | CTCs | CTCs | CTCs | CTCs | CTCs | CTCs | CTCs |
| | | | T +ve | CD45⁺ | CD45⁺ | CD45⁺ | CD45⁺ | CD45⁺ | CD45⁺ | CD45⁺ | T +ve | T +ve |
| | | | CSCs | T +ve | T +ve | T +ve | T +ve | T +ve | T +ve | T +ve | CSCs | CSCs |
| | | | T −ve | CSCs | CSCs | CSCs | CSCs | CSCs | CSCs | CSCs | T −ve | T −ve |
| | | | | T −ve | T −ve | T −ve | T −ve | T −ve | T −ve | T −ve | | |
| 2 | Drug | — | — | — | 40 | 40 | 40 | 40 | 200 | 200 | 100 | 100 |
| | conc | | | | μg/ml | μg/ml | μg/ml | μg/ml | μg/ml | μg/ml | ng/ml | ng/ml |
| | 2 | | | | | | | | | | | |
| | Cells | — | CTCs | CTCs | CTCs | CTCs | CTCs | CTCs | CTCs | CTCs | CTCs | CTCs |
| | | | T +ve | CD45⁺ | CD45⁺ | CD45⁺ | CD45⁺ | CD45⁺ | CD45⁺ | CD45⁺ | T +ve | T +ve |
| | | | CSCs | T +ve | T +ve | T +ve | T +ve | T +ve | T +ve | T +ve | CSCs | CSCs |
| | | | T −ve | CSCs | CSCs | CSCs | CSCs | CSCs | CSCs | CSCs | T −ve | T −ve |
| | | | | T −ve | T −ve | T −ve | T −ve | T −ve | T −ve | T −ve | | |
| 3 | Drug | — | — | — | 100 | 100 | 80 | 80 | 400 | 400 | 200 | 200 |
| | conc | | | | μg/ml | μg/ml | μg/ml | μg/ml | μg/ml | μg/ml | ng/ml | ng/ml |
| | 3 | | | | | | | | | | | |
| | Cells | — | CTCs | CTCs | CTCs | CTCs | CTCs | CTCs | CTCs | CTCs | CTCs | CTCs |
| | | | T +ve | CD45⁺ | CD45⁺ | CD45⁺ | CD45⁺ | CD45⁺ | CD45⁺ | CD45⁺ | T +ve | T +ve |
| | | | CSCs | T +ve | T +ve | T +ve | T +ve | T +ve | T +ve | T +ve | CSCs | CSCs |
| | | | T −ve | CSCs | CSCs | CSCs | CSCs | CSCs | CSCs | CSCs | T −ve | T −ve |
| | | | | T −ve | T −ve | T −ve | T −ve | T −ve | T −ve | T −ve | | |
| 4 | Drug | — | — | — | 160 | 160 | 160 | 160 | 800 | 800 | 400 | 400 |
| | conc | | | | μg/ml | μg/ml | μg/ml | μg/ml | μg/ml | μg/ml | ng/ml | ng/ml |
| | 4 | | | | | | | | | | | |
| | Cells | — | CTCs | CTCs | CTCs | CTCs | CTCs | CTCs | CTCs | CTCs | CTCs | CTCs |
| | | | T +ve | CD45⁺ | CD45⁺ | CD45⁺ | CD45⁺ | CD45⁺ | CD45⁺ | CD45⁺ | T +ve | T +ve |
| | | | CSCs | T +ve | T +ve | T +ve | T +ve | T +ve | T +ve | T +ve | CSCs | CSCs |
| | | | T −ve | CSCs | CSCs | CSCs | CSCs | CSCs | CSCs | CSCs | T −ve | T −ve |
| | | | | T −ve | T −ve | T −ve | T −ve | T −ve | T −ve | T −ve | | |

| | | Treatment | | | | | | | |
|---|---|---|---|---|---|---|---|---|---|
| | | | | | | 5 | | | |
| | | | | | | Fluoro- | | Beva- | |
| Row | | Paclitaxel | | Irinotecan | | Uracil | | cizumab | |
| A: Assay layout for Example 9 - blood sample | | | | | | | | | |
| 1 | Drug | 0.05 | 0.05 | 5 | 5 | 250 | 250 | 1 | 1 |
| | conc | μg/ml | μg/ml | μg/ml | μg/ml | ng/ml | ng/ml | μg/ml | μg/ml |
| | 1 | | | | | | | | |
| | Cells | CTCs | CTCs | CTCs | CTCs | CTCs | CTCs | CTCs | CTCs |
| | | T −ve | T −ve | T −ve | T −ve | T −ve | T −ve | T −ve | T −ve |
| 2 | Drug | 0.1 | 0.1 | 10 | 10 | 500 | 500 | 2 | 2 |
| | conc | μg/ml | μg/ml | μg/ml | μg/ml | ng/ml | ng/ml | μg/ml | μg/ml |
| | 2 | | | | | | | | |
| | Cells | CTCs | CTCs | CTCs | CTCs | CTCs | CTCs | CTCs | CTCs |
| | | T −ve | T −ve | T −ve | T −ve | T −ve | T −ve | T −ve | T −ve |
| 3 | Drug | 0.2 | 0.2 | 25 | 25 | 1000 | 1000 | 4 | 4 |
| | conc | μg/ml | μg/ml | μg/ml | μg/ml | ng/ml | ng/ml | μg/ml | μg/ml |
| | 3 | | | | | | | | |
| | Cells | CTCs | CTCs | CTCs | CTCs | CTCs | CTCs | CTCs | CTCs |
| | | T −ve | T −ve | T −ve | T −ve | T −ve | T −ve | T −ve | T −ve |
| 4 | Drug | 1 | 1 | 50 | 50 | 2000 | 2000 | 8 | 8 |
| | conc | μg/ml | μg/ml | μg/ml | μg/ml | ng/ml | ng/ml | μg/ml | μg/ml |
| | 4 | | | | | | | | |
| | Cells | CTCs | CTCs | CTCs | CTCs | CTCs | CTCs | CTCs | CTCs |
| | | T −ve | T −ve | T −ve | T −ve | T −ve | T −ve | T −ve | T −ve |
| B: Assay layout for Example 9 - tissue sample | | | | | | | | | |
| 1 | Drug | 0.05 | 0.05 | 5 | 5 | 250 | 250 | 1 | 1 |
| | conc | μg/ml | μg/ml | μg/ml | μg/ml | ng/ml | ng/ml | μg/ml | μg/ml |
| | 1 | | | | | | | | |
| | Cells | CTCs | CTCs | CTCs | CTCs | CTCs | CTCs | CTCs | CTCs |
| | | T +ve | T +ve | T +ve | T +ve | T +ve | T +ve | T +ve | T +ve |
| | | CSCs | CSCs | CSCs | CSCs | CSCs | CSCs | CSCs | CSCs |
| | | T −ve | T −ve | T −ve | T −ve | T −ve | T −ve | T −ve | T −ve |
| 2 | Drug | 0.1 | 0.1 | 10 | 10 | 500 | 500 | 2 | 2 |
| | conc | μg/ml | μg/ml | μg/ml | μg/ml | ng/ml | ng/ml | μg/ml | μg/ml |
| | 2 | | | | | | | | |
| | Cells | CTCs | CTCs | CTCs | CTCs | CTCs | CTCs | CTCs | CTCs |
| | | T +ve | T +ve | T +ve | T +ve | T +ve | T +ve | T +ve | T +ve |
| | | CSCs | CSCs | CSCs | CSCs | CSCs | CSCs | CSCs | CSCs |
| | | T −ve | T −ve | T −ve | T −ve | T −ve | T −ve | T −ve | T −ve |
| 3 | Drug | 0.2 | 0.2 | 25 | 25 | 1000 | 1000 | 4 | 4 |
| | conc | μg/ml | μg/ml | μg/ml | μg/ml | ng/ml | ng/ml | μg/ml | μg/ml |
| | 3 | | | | | | | | |
| | Cells | CTCs | CTCs | CTCs | CTCs | CTCs | CTCs | CTCs | CTCs |
| | | T +ve | T +ve | T +ve | T +ve | T +ve | T +ve | T +ve | T +ve |
| | | CSCs | CSCs | CSCs | CSCs | CSCs | CSCs | CSCs | CSCs |
| | | T −ve | T −ve | T −ve | T −ve | T −ve | T −ve | T −ve | T −ve |
| 4 | Drug | 1 | 1 | 50 | 50 | 2000 | 2000 | 8 | 8 |
| | conc | μg/ml | μg/ml | μg/ml | μg/ml | ng/ml | ng/ml | μg/ml | μg/ml |
| | 4 | | | | | | | | |
| | Cells | CTCs | CTCs | CTCs | CTCs | CTCs | CTCs | CTCs | CTCs |
| | | T +ve | T +ve | T +ve | T +ve | T +ve | T +ve | T +ve | T +ve |
| | | CSCs | CSCs | CSCs | CSCs | CSCs | CSCs | CSCs | CSCs |
| | | T −ve | T −ve | T −ve | T −ve | T −ve | T −ve | T −ve | T −ve |

CTCs = circulating tumour cells;
CD45^{⁺} = CD45⁺ cells;
T −ve = tumour marker negative cells;
T +ve = tumour marker positive cells;
CSCs = cancer stem cells

Interpreting these drug response scores, the conclusions are shown in Table 30:

TABLE 30
Drug sensitivity as calculated by the assay
Based on Drug Response Scores the following interpretation is made:

| | | Blood | Tissue |
|---|---|---|---|
| 1 | Pembrolizumab | S+ | S+ |
| 2 | Nivolumab | R | S+ |
| 3 | Atezolizumab | S++ | S+ |
| 4 | Lenvatinib | S+ | S+ |
| 5 | Albumin bound Paclitaxel | S+ | R |
| 6 | Irinotecan | S+ | S+ |
| 7 | 5FU | S+ | S+ |
| 8 | Bevacizumab | R | R |

Under laboratory conditions, all immunotherapy drugs (Pembrolizumab; Nivolumab; Atezolizumab) show effectiveness (S+) on Cancer Cells from tissue sample. Lenvatinib, Irinotecan and 5 FluoroUracil also show effectiveness (S+) on Cancer cells from Tissue Sample. Under laboratory conditions, Atezolizumab (S++) and Pembrolizumab (S+) are effective on Circulating tumour cells. Lenvatinib, Paclitaxel, Irinotecan and 5 FluoroUracial show effectiveness (S+) on Circulating tumour cells under laboratory conditions.

### Example 10-Determination of Treatment for Patient with Osteosarcoma

Tissue samples from a patient with osteosarcoma (patient previously given methotrexate and cisplatin+ doxorubicin) were received on 5 Jan. 2022 and tested using a 5 drug panel.

Chemotherapeutic agents were tested as in Table 31.

The drug response scores for this panel are shown in Table 32:

TABLE 32
Drug response scores for the 5 drug panel

| No. | Condition | conc. 1 | conc. 2 | conc. 3 | conc. 4 |
|---|---|---|---|---|---|
| 1 | Etop | 0.4829 | 0.0653 | −0.4472 | 0.0955 |
| 2 | DTIC | −0.7928 | 0.4035 | −0.0520 | 0.1714 |
| 3 | Ifos | 0.3183 | 0.8776 | 0.1237 | −0.8671 |
| 4 | Doce | −0.5113 | 1.0268 | 0.6693 | −0.6056 |
| 5 | Irina | −0.0303 | −0.1454 | 0.2672 | −0.0433 |

Etop = Etoposide;
DTIC = Dacarbazine;
Ifos = Ifosfamide;
Doce = Docetaxel;
Irina = Irinotecan

TABLE 31
Assay layout for Example 10

| | | Treatment | | | | | | | | | |
|---|---|---|---|---|---|---|---|---|---|---|---|
| | | | Blank | | | | | | | | |
| Row | | Blank | cells | Etoposide | | Dacarbazine | | | | Ifosfamide | |
| 1 | Drug | — | — | 1 μg/ml | 1 μg/ml | 1.5 | μg/ml | 1.5 | μg/ml | 0.125 | μg/ml |
| | conc 1 | | | | | | | | | | |
| | Cells | — | CTCs | CTCs | CTCs | CTCs | | CTCs | | CTCs | |
| | | | T +ve | T +ve | T +ve | T +ve | | T +ve | | T +ve | |
| | | | CSCs | CSCs | CSCs | CSCs | | CSCs | | CSCs | |
| | | | T −ve | T −ve | T −ve | T −ve | | T −ve | | T −ve | |
| 2 | Drug | — | — | 4 μg/ml | 4 μg/ml | 3 | μg/ml | 3 | μg/ml | 0.25 | μg/ml |
| | conc 2 | | | | | | | | | | |
| | Cells | — | CTCs | CTCs | CTCs | CTCs | | CTCs | | CTCs | |
| | | | T +ve | T +ve | T +ve | T +ve | | T +ve | | T +ve | |
| | | | CSCs | CSCs | CSCs | CSCs | | CSCs | | CSCs | |
| | | | T −ve | T −ve | T −ve | T −ve | | T −ve | | T −ve | |
| 3 | Drug | — | — | 20 μg/ml | 20 μg/ml | 6 | μg/ml | 6 | μg/ml | 5 | μg/ml |
| | conc 3 | | | | | | | | | | |
| | Cells | — | CTCs | CTCs | CTCs | CTCs | | CTCs | | CTCs | |
| | | | T +ve | T +ve | T +ve | T +ve | | T +ve | | T +ve | |
| | | | CSCs | CSCs | CSCs | CSCs | | CSCs | | CSCs | |
| | | | T −ve | T −ve | T −ve | T −ve | | T −ve | | T −ve | |
| 4 | Drug | — | — | 100 μg/ml | 100 μg/ml | 12 | μg/ml | 12 | μg/ml | 10 | μg/ml |
| | conc 4 | | | | | | | | | | |

| | | | | | | | |
|---|---|---|---|---|---|---|---|
| Cells | — | CTCs | CTCs | CTCs | CTCs | CTCs | CTCs |
| | | T +ve | T +ve | T +ve | T +ve | T +ve | T +ve |
| | | CSCs | CSCs | CSCs | CSCs | CSCs | CSCs |
| | | T −ve | T −ve | T −ve | T −ve | T −ve | T −ve |

| | | Treatment | | | | | | | |
|---|---|---|---|---|---|---|---|---|---|
| Row | | Ifosfamide | | Docetaxel | | | | Irinotecan | |
| 1 | Drug | 0.125 | μg/ml | 0.02 | μg/ml | 0.02 | μg/ml | 5 μg/ml | 5 μg/ml |
| | conc 1 | | | | | | | | |
| | Cells | CTCs | | CTCs | | CTCs | | CTCs | CTCs |
| | | T +ve | | T +ve | | T +ve | | T +ve | T +ve |
| | | CSCs | | CSCs | | CSCs | | CSCs | CSCs |
| | | T −ve | | T −ve | | T −ve | | T −ve | T −ve |
| 2 | Drug | 0.25 | μg/ml | 0.2 | μg/ml | 0.2 | μg/ml | 10 μg/ml | 10 μg/ml |
| | conc 2 | | | | | | | | |
| | Cells | CTCs | | CTCs | | CTCs | | CTCs | CTCs |
| | | T +ve | | T +ve | | T +ve | | T +ve | T +ve |
| | | CSCs | | CSCs | | CSCs | | CSCs | CSCs |
| | | T −ve | | T −ve | | T −ve | | T −ve | T −ve |
| 3 | Drug | 5 | μg/ml | 2 | μg/ml | 2 | μg/ml | 25 μg/ml | 25 μg/ml |
| | conc 3 | | | | | | | | |
| | Cells | CTCs | | CTCs | | CTCs | | CTCs | CTCs |
| | | T +ve | | T +ve | | T +ve | | T +ve | T +ve |
| | | CSCs | | CSCs | | CSCs | | CSCs | CSCs |
| | | T −ve | | T −ve | | T −ve | | T −ve | T −ve |
| 4 | Drug | 10 | μg/ml | 10 | μg/ml | 10 | μg/ml | 50 μg/ml | 50 μg/ml |
| | conc 4 | | | | | | | | |

| | | | | | |
|---|---|---|---|---|---|
| Cells | CTCs | CTCs | CTCs | CTCs | CTCs |
| | T +ve | T +ve | T +ve | T +ve | T +ve |
| | CSCs | CSCs | CSCs | CSCs | CSCs |
| | T −ve | T −ve | T −ve | T −ve | T −ve |

CTCs = circulating tumour cells;
T −ve = tumour marker negative cells;
T +ve = tumour marker positive cells;
CSCs = cancer stem cells

Interpreting these drug response scores, the conclusions are shown in Table 33:

TABLE 33
Drug sensitivity as calculated by the assay
Based on Drug Response Scores the following interpretation is made:

| | | |
|---|---|---|
| 1 | Etoposide | Sensitive (S+) |
| 2 | DTIC (Dacarbazine) | Sensitive (S++) |
| 3 | Ifosfamide (Holoxan) | Sensitive (S+) |
| 4 | Docetaxel | Resistant |
| 5 | Irinotecan | Sensitive (S++) |

Under laboratory conditions, Etoposide, DTIC (Dacarbazine), Ifosfamide and Irinotecan show effectiveness on the cancer cells derived from the tissue sample. Under laboratory conditions, cancer cells derived from tissue sample are deemed resistant to docetaxel.

1. A method of predicting responsiveness of a subject having cancer to one or more immunotherapeutic agent and/or one or more chemotherapeutic agent, said method comprising the steps of:
(a) providing a first and a second biocompatible scaffold;
(b) providing at least one population of CD45⁺ cells from the subject and at least one population of circulating tumour cells (CTCs) from the subject;
(c) providing at least one chemotherapeutic agent and at least one immunotherapeutic agent;
(d) plating onto the first biocompatible scaffold the at least one population of CD45⁺ cells and the at least one population of circulating tumour cells (CTCs);
(e) contacting the plated at least one population of CD45⁺ cells and at least one population of circulating tumour cells (CTCs) with the at least one immunotherapeutic agent;
(e) plating onto the second biocompatible scaffold the at least one population of CTCs;
(f) contacting the at least one population of CTCs with the at least one chemotherapeutic agent; and
(g) determining the efficacy of the at least one immunotherapeutic agent and/or the at least one therapeutic agent against the CTCs.

2. (canceled)

3. The method of claim 1, further comprising the steps of:
(h) providing a third biocompatible scaffold;
(i) providing at least one population of tumour marker⁺ cells and at least one population of cancer stem cells (CSCs);
(j) providing at least one immunotherapeutic agent;
(k) plating the at least one population of tumour marker⁺ cells and/or at least one population of CSCs on the first, the second and/or the third biocompatible scaffold;
(l) contacting at least one population of tumour marker⁺ cells and/or CSCs with the at least one immunotherapeutic agent; and
(m) determining the efficacy of the at least one immunotherapeutic agent against the tumour marker⁺ cells and/or CSCs,
wherein optionally the at least one immunotherapeutic agent is the same at least one immunotherapeutic agent as used in step (e).

4.-5. (canceled)

6. The method of claim 1, wherein the CD45⁺ cells and/or CTCs have been isolated from a blood sample from the subject.

7. The method of claim 3, wherein the CSCs, tumour marker⁺ cells and/or CD45⁺cells have been isolated from a sample of cancerous tissue from the subject.

8. The method claim 7, wherein the CD45⁺ cells, CTCs, CSCs, and/or tumour marker⁺ cells are isolated from the sample by a method comprising using magnetic activated cell sorting (MACS), fluorescence activated cell sorting (FACS), flow cytometry, or buoyancy activated cell sorting (BACS).

9. The method of claim 1, wherein the CD45⁺ cells:
(a) are haematopoietic in origin;
(b) originate from the bone marrow; and/or
(c) are lymphoid.

10. (canceled)

11. The method of claim 1, wherein the CD45⁺ cells and/or the CTCs are less than 20 μm in diameter.

12. The method of claim 3, wherein:
(i) the CTCs are EpCAM⁺CD45⁻cells;
(ii) the CSCs are CD133⁺ cells;
(iii) the tumour marker cells are (1) CD31⁺, CD45⁺ and/or Gly-A⁺; and/or (2) can be bound by an anti-fibroblast antibody; and/or
(iv) the tumour marker⁺ cells are the remaining cells from the tissue sample that are not tumour marker cells.

13. The method of claim 3, wherein the CSCs, and/or tumour marker⁺ cells are present in essentially the same proportions as in the cancerous tissue sample.

14. The method of claim 1, wherein the cancer is a solid cancer.

15. A method according to claim 14, wherein the solid cancer is selected from lung cancer, breast cancer, colon cancer, prostate cancer, melanoma (skin cancer), kidney cancer (renal cell carcinoma), head and neck cancer (squamous cell carcinoma), pancreatic cancer, brain or CNS cancer, bladder cancer, oesophageal cancer, cancer of unknown primary, ovarian cancer, stomach cancer, liver cancer, thyroid cancer and uterine cancer.

16. The method of claim 1, wherein:
(i) all steps comprising contacting a cell population with at least one immunotherapeutic agent and/or at least one chemotherapeutic agent are carried out simultaneously; or
(ii) the steps comprising contacting a cell population with at least one immunotherapeutic agent and/or at least one chemotherapeutic agent are carried out sequentially.

17. The method of claim 1, wherein efficacy of the at least one immunotherapeutic agent and/or the at least one chemotherapeutic agent is determined by cytotoxicity and/or cell viability assay.

18. (canceled)

19. The method according to claim 1, wherein the biocompatible scaffold comprises or consists of a layer of biocompatible electrospun polymer fibers.

20. The method according to claim 19, wherein the electrospun polymer fibers comprise a polymer selected from the group consisting of polyethylene terephthalate, silicone, polyurethane, polycarbonate, polyether ketone, polycaprolactone, polylactic acid, polyglycolic acid, collagen, gelatin, fibronectin, hyaluronic acid, and combinations thereof.

21. The method of claim 1, wherein the time taken to predict responsiveness of a subject having cancer to one or more immunotherapeutic agent and one or more chemotherapeutic agent is 15 days or less, optionally 10 days or less.

22. The method of claim 1, which has a success rate of at least 75%, preferably at least 80%, more preferably at least 90% of correctly predicting responsiveness of a subject having cancer to one or more immunotherapeutic agent and one or more chemotherapeutic agent.

23. A kit comprising one of claims 1 to 22, which comprises:
(i) a biocompatible scaffold, optionally attached to an inert support;
(ii) CD45⁺ cells, CTCs, CSCs, tumour marker⁺ cells and/or tumour marker cells; and/or
one or more agents for determining cell viability and/or cytotoxicity.

24.-25. (canceled)

26. A method of treating cancer in a subject in need thereof, said method comprising:
(a) selecting one or more immunotherapeutic agents and/or one or more chemotherapeutic agents for treating the cancer in the subject in need thereof by determining therapeutic responsiveness of the subject in need thereof to one or more immunotherapeutic agents and one or more chemotherapeutic agents, the selecting method comprising the steps of:
(i) contacting at least one population of CD45⁺ cells and circulating tumour cells (CTCs) from said subject with at least one immunotherapeutic agent;
(ii) contacting at least one population of circulating tumour cells (CTCs) from said subject with at least one chemotherapeutic agent;
(ii) determining the efficacy of the at least one immunotherapeutic agent and at least one therapeutic agent against the CTCs; and
(iv) selecting one or more immunotherapeutic agents and/or one or more chemotherapeutic agents on the basis of the subject in need thereof's responsiveness to the one or more immunotherapeutic agent and/or one or more chemotherapeutic agent; and
(b) administering an effective amount of the one or more immunotherapeutic agents and/or one or more chemotherapeutic agents as selected in step (a) to the subject in need thereof.
